(19) United States
(12) Patent Application Publication
(10) Pub. No.: US 2026/0033985 A1
(43) Pub. Date: Feb. 5, 2026

(54) METHODS AND DEVICES FOR HEATING IMPLANTS

(71) Applicants: Jimmy Shah, Brooklyn, NY (US); Nick N. Patel, Wilmington, DE (US)

(72) Inventors: Jimmy Shah, Brooklyn, NY (US); Nick N. Patel, Wilmington, DE (US)

(21) Appl. No.: 19/290,254

(22) Filed: Aug. 4, 2025

Related U.S. Application Data
(60) Provisional application No. 63/679,201, filed on Aug. 5, 2024.

(51) Int. Cl. A61F 7/12; A61B 17/88; A61F 2/30; A61F 2/46; A61F 7/00

(52) U.S. Cl. CPC A61F 7/12; A61B 17/8836; A61B 17/8847; A61F 2/461; A61F 2002/30719; A61F 2002/465; A61F 2007/0071; A61F 2007/0087; A61F 2007/0096; A61F 2007/126

(57) ABSTRACT

Methods, systems, and devices for heating orthopedic implants are disclosed. The system includes an orthopedic implant receiving heat from a heating device. The heating device includes a heating element and a casing supporting or housing the heating element. A control unit provides precise and reliable operation of the heating element by managing power delivery, regulating temperature, and ensuring safe and effective functioning of the heating device during orthopedic surgical procedures. The casing is shaped and dimensioned for engagement with an orthopedic implant. Briefly, according to the method, resistive heat is applied to the orthopedic implant by first accessing an exposed implant surface, applying a retractor instrument to aid in exposure of the orthopedic implant and protection of surrounding soft tissues, attaching a heating device to the exposed implant surface, and activating the heating device.

### CROSS-REFERENCE TO RELATED APPLICATIONS

The present application claims the benefit of U.S. Provisional Patent Application Ser. No. 63/679,201, entitled “METHODS AND DEVICES FOR HEATING IMPLANTS,” filed Aug. 5, 2024, which is incorporated herein by reference.

### BACKGROUND OF THE INVENTION

### 1. Field of the Invention

The invention pertains to medical devices used in orthopedic surgery. More specifically, the invention relates to devices designed for heating implants within the body to facilitate their removal, clear bacterial infections, and accelerate the curing process of bone cement.

### 2. Related Art

Orthopedic surgery often involves the use of implants to repair or replace damaged joints and bones. These implants, including knee, hip, ankle, shoulder, and elbow prostheses, are critical for restoring function and reducing pain for patients with severe joint and bone conditions. However, various challenges are associated with the use and maintenance of these implants.

One significant challenge is the removal of well-fixated implants during revision surgeries. These implants can be securely bonded to the surrounding bone either through bone cement or biological integration, making extraction difficult and time-consuming. The current methods for implant removal often require extensive mechanical force or specialized cutting tools, which can cause additional bone damage and increase morbidity. Additionally, inadequate release of the implant from the attached bone during removal can lead to significant irreversible bone damage. Additionally, the added surgical time for implant removal with current methods can lead to increased cost to the healthcare system and increased anesthesia related risks for the patient.

Another major issue associated with implants within the body is infection. As they involve foreign material within the body, implants can be prone to infection. Peri-implant infection is typically difficult to clear without further intervention for multiple reasons. The body's immune system cannot as effectively clear infection from implants due to the fact that they are foreign material and not as susceptible to the immune response. Bacteria can adhere to implant surfaces and form biofilm. This can occur acutely or chronically after the implant placement within the body. The biofilm matrix creates somewhat of a physical barrier, making it difficult for immune cells or antibiotics to penetrate. Biofilms can be resistant to standard antibiotic treatment and surgical debridement. Peri-implant infections are also difficult to clear because bacteria can embed within the porous surface of implant materials or within the component junctions or interfaces. Existing methods to clear infection associated with implants are often inadequate, leading to recurrent or persistent infection, need for additional surgeries, and poor patient outcomes.

Furthermore, a common form of implant fixation to bone involves the utilization of bone cement. This is frequently utilized in procedures such as joint replacement. The curing process of bone cement is critical for the stability and longevity of the implant. However, this process can be time-consuming, prolonging the duration of the surgical procedure thus increasing the risk to the patient and cost to the healthcare system. Additionally, if the cement does not adequately cure prior to joint or extremity manipulation, there can be risk for improper implant fixation and alignment.

### SUMMARY OF THE INVENTION

In one aspect a system for heating orthopedic implants comprises a heating device comprising a heating element and a casing supporting or housing the heating element, the casing being shaped and dimensioned for engagement with an orthopedic implant. The system also includes a control unit providing precise and reliable operation of the heating element by managing power delivery, regulating temperature, and ensuring the safe and effective functioning of the device during orthopedic surgical procedures.

In some embodiments the system further includes an attachment mechanism.

In some embodiments the heating element comprises a resistance heating wire.

In some embodiments the heating wire has a serpentine or spiral configuration.

In some embodiments the system includes a temperature sensor.

In some embodiments the temperature sensor is coupled to the casing.

In some embodiments the temperature sensor is shaped and dimensioned for attachment to the orthopedic implant.

In some embodiments the heating element is adapted to heat the orthopedic implant to 60° C. to 140° C.

In some embodiments the heating element operates at temperatures above 140° C.

In some embodiments the heating element operates at a temperature of 100° C. to 150° C.

In some embodiments the heating element operates at a temperature of 60° C. to 100° C.

In some embodiments the heating element operates at a temperature of 40° C. to 100° C.

In some embodiments the casing comprises medical-grade silicone rubber, polyimide film, PTFE, stainless steel, aluminum, or medical-grade PEEK, or a combination thereof.

In some embodiments the casing comprises stainless steel, aluminum, or medical-grade PEEK.

In some embodiments one side of the casing is shaped and dimensioned to contact exposed surfaces of the orthopedic implant and is made from a thermal conductor to ensure efficient heat transfer directly into the orthopedic implant.

In some embodiments the thermal conductor comprises medical-grade aluminum, copper, medical-grade silicone rubber, polyimide film, PTFE, stainless steel, or medical-grade PEEK, or a combination thereof.

In some embodiments an opposing side of the casing is constructed from a thermal insulator.

In some embodiments the thermal insulator is silicone rubber, PTFE, or medical-grade PEEK, or a combination thereof.

In some embodiments the casing includes multiple heating zones for heating a single orthopedic implant or for heating multiple orthopedic implants.

In some embodiments the control unit operates in a manual mode and a preprogrammed heating cycle mode.

In some embodiments the control unit comprises a feedback loop that adjusts the power output to maintain a target temperature.

In some embodiments the heating element is embedded or encapsulated within the casing.

In some embodiments the heating element is on an exposed surface of the casing and can be directly exposed to the implant surface.

In some embodiments the casing is adapted to be trimmed or modified to the desired shape which will not disrupt or damage the heating element.

In some embodiments the casing incorporates multiple heating zones, each tailored to contact distinct implant surfaces.

In some embodiments the casing is structured for simultaneously heating both femoral and tibial components in a knee revision procedure.

In some embodiments the heating element includes a first heating element and a second heating element. The first heating element and the second heating element are housed within a casing shaped and dimensioned to contact both a tibial component and the femoral component.

In some embodiments the casing includes a casing body that comprises a first member shaped and dimensioned for contact with the tibial component and a second member shaped and dimensioned for contact with a femoral component.

In some embodiments the first member is substantially rectangular in shape and the second member is substantially C-shaped.

In another aspect a heating device includes a heating element comprising a resistance heating wire and a casing for supporting or housing the heating element. The heating element includes a first heating element and a second heating element. The casing includes a casing body comprising a first member and a second member shaped and dimensioned for engagement with an orthopedic implant, the first heating element and the second heating element are respectively housed within the first member and the second member.

In some embodiments the first member is shaped and dimensioned for contact with a tibial component and the second member is shaped and dimensioned for contact with a femoral component.

In some embodiments the heating device includes a temperature sensor.

In some embodiments the first member and the second member of the casing define multiple heating zones, each tailored to contact distinct implant surfaces.

In some embodiments the heating device further includes a handle extending from the casing allowing a user to conveniently position the heating device upon the orthopedic implant.

In some embodiments the casing is structured for simultaneously heating both femoral and tibial components in a knee revision procedure.

In some embodiments the first member of the casing is shaped and dimensioned for contact with the tibial component and the second member of the casing is shaped and dimensioned for contact with a femoral component.

In another aspect a system for heating orthopedic implants comprises an orthopedic implant, a heating element, a casing supporting or housing the heating element, the casing being shaped and dimensioned for engagement with an orthopedic implant, and a control unit providing precise and reliable operation of the heating element by managing power delivery, regulating temperature, and ensuring the safe and effective functioning of the device during orthopedic surgical procedures.

In some embodiments the orthopedic implant comprises a conductive material.

In some embodiments the conductive material is silver, copper, gold, aluminum, tungsten, iron, platinum, tin, titanium, cobalt-chromium alloy, stainless steel, zirconium, nickel, and combinations thereof.

In some embodiments the orthopedic implant is a knee implant.

In some embodiments the orthopedic implant is a hip implant.

In some embodiments the orthopedic implant comprises exposed surfaces that are not in direct contact with bone or cement and non-exposed surfaces that remain in direct contact with cement, bone, or are embedded within the bone structure.

In some embodiments the non-exposed surfaces are either surrounded by a layer of bone cement which acts as a filler and locking agent between the orthopedic implant and the bone or surrounded directly by bone.

In a further aspect a method of applying resistive heat to an orthopedic implant comprises accessing an exposed implant surface, applying a retractor instrument to aid in exposure of the orthopedic implant and protection of surrounding soft tissues, attaching a heating device to the exposed implant surface, and activating the heating device.

In some embodiments the method further includes removing modular components of the orthopedic implant to fully expose underlying implant surfaces.

In some embodiments the heating device includes a heating element and a casing supporting or housing the heating element, the casing being shaped and dimensioned for engagement with an orthopedic implant. The system also includes a control unit providing precise and reliable operation of the heating element by managing power delivery, regulating temperature, and ensuring the safe and effective functioning of the device during orthopedic surgical procedures.

In some embodiments the system further includes temperature sensors, and the temperature sensors are positioned on the orthopedic implant and/or the casing.

In some embodiments activating the heating device includes activating the control unit.

In some embodiments the control unit heats the heating element to apply sufficient heat to bone cement so that the bone cement reaches a temperature between 60° C. and 140° C.

In some embodiments the control unit heats the heating element to apply sufficient heat to heat an exposed surface of the orthopedic implant to between 100° C. and 160° C.

In some embodiments the control unit heats the heating element to apply sufficient heat to heat the orthopedic implant to between 40° C. and 120° C. to reduce bacterial load from the surface of the orthopedic implant.

In some embodiments the control unit heats the heating element to apply sufficient heat to cure bone cement.

Other objects and advantages of the present invention will become apparent from the following detailed description when viewed in conjunction with the accompanying drawings, which set forth certain embodiments of the invention.

### BRIEF DESCRIPTION OF DRAWINGS

Aspects of the present disclosure are illustrated by way of example and are not limited by the accompanying figures with like reference numbers indicating like elements.

FIG. 1 depicts an oblique view of a heating device for a tibial implant according to a first embodiment.

FIGS. 2A-B depict oblique views of a heating device, according to a second embodiment, on a femoral implant.

FIG. 3 depicts an oblique view of a heating device for a femoral implant according to the second embodiment.

FIGS. 4A-B depict views of a multi-part heating device, according to a third embodiment, on a femoral implant. FIG. 4A depicts an oblique view. FIG. 4B depicts an underside view.

FIGS. 5A-B depict a side view of the femur, femoral knee implant, and heating device according to the second embodiment. FIG. 5A depicts the heating device on a femoral implant located on a transparent femur. FIG. 5B depicts an exploded lateral view of the femur, femoral knee implant, and heating device.

FIGS. 6A-C depict a side view of the heating device, according to a fourth embodiment, conforming to the complex surface shape of the femoral knee implant. FIG. 6A depicts an un-bent, relatively flat heating device being applied to the femoral knee implant. FIG. 6B depicts the flat heating device being bent and shaped to match the surface geometry of the femoral knee implant. FIG. 6C depicts the heating device fully conformed and attached to the surface of the knee implant.

FIGS. 7A-B depict views of the tibia, tibial knee implant, and tibial heating device according to the first embodiment. FIG. 7A depicts a back view of the heating device positioned within the tibial implant tray. The tibial implant is positioned within a transparent tibia. FIG. 7B depicts a front view of the heating device positioned within the tibial implant tray. The tibial implant is positioned within a transparent tibia.

FIGS. 8A-B depict cross-section views of the tibial knee implant and the tibial implant heating device according to the first embodiment. FIG. 8A depicts the heating device being lowered into the recessed tray located on the tibial knee implant. FIG. 8B depicts the heating device positioned within the recessed tray located on the tibial knee implant.

FIGS. 9A-B depict oblique views of the tibial implant heating device according to the first embodiment. FIG. 9A depicts a bottom-oblique view of the heating device. FIG. 9B depicts an exploded view of the components of the heating device.

FIGS. 10A-B depict cross-sectional side views of the total knee implant with a heating device according to the first embodiment. FIG. 10A depicts a transparent view of the femoral and tibial knee implants within the bones, with a heating device positioned in between and in direct contact with both components of the knee implant. FIG. 10B depicts a cross-sectional view of how the components of the heating device are in direct contact with both the femoral and tibial knee implant simultaneously.

FIGS. 11A-B depict oblique views of the heating devices according to the first and second embodiments, respectively, including a temperature sensor mounted on the knee implant components. FIG. 11A depicts the heating device according to the second embodiment and a temperature sensor positioned on a femoral knee implant. FIG. 11B depicts a heating device according to the first embodiment and a temperature sensor positioned on a tibial knee implant.

FIG. 12 depicts the control unit, power source, and various heating devices.

FIGS. 13A-C depict different shapes of the heating device. FIG. 13A shows a heating device to be used on a femoral knee implant. FIG. 13B depicts a T-shaped heating device. FIG. 13C depicts a fan-shaped heating device capable of conforming to a wide range of orthopedic implant shapes.

FIG. 14 depicts the bone cement layer surrounding a tibial knee implant.

FIG. 15 depicts a fifth embodiment disclosing a single heating pad applying heat simultaneously to both the femoral and tibial implants within a knee.

FIG. 16 is an exploded view showing a system employing a sixth embodiment of a heating device with a casing incorporating multiple heating zones, each tailored to contact distinct implant surfaces while being powered and controlled through a single or modular system.

FIGS. 17A-17F showing the steps associated with the use of the heating device shown in FIG. 16.

FIG. 18 is a perspective view of an alternate embodiment of the heating device disclosed in FIG. 16, wherein the heating device includes a rechargeable battery.

FIG. 19 is a perspective view of an alternate embodiment of the heating device disclosed in FIG. 16, wherein the heating element is exposed and not embedded within the casing of the heating device.

FIGS. 20A & 20B disclose alternate embodiments of a heating device that may be cut.

FIG. 21 is a perspective view of an alternate embodiment of the heating device disclosed in FIG. 16, wherein the heating device includes a handle integrally formed therewith.

FIGS. 22A & 22B are a perspective view a cross sectional view showing an alternate embodiment of the heating device disclosed in FIG. 16, wherein the heating device includes a deformable/soft/spongy layer on the inner surface thereof.

FIG. 23 is a perspective view of an alternate embodiment of the heating device disclosed in FIG. 16, wherein the heating device includes magnets providing an effective attachment solution for metallic orthopedic implants.

FIG. 24 is a perspective view of an alternate embodiment of the heating device disclosed in FIG. 16, wherein the heating device possessing enough flexibility to deform slightly under manual pressure but sufficient stiffness to “snap” into place once positioned.

FIGS. 25A & 25B are side views showing the use of the heating device shown in FIG. 24.

FIGS. 26A & 26B show a heating device in accordance with the present invention used in conjunction with a hip implant, specifically, in conjunction with the femoral component and the acetabulum components.

### DETAILED DESCRIPTION OF THE EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as a basis for teaching one skilled in the art how to make and/or use the invention.

Various embodiments are disclosed herein. It should be appreciated that similar reference numerals are used for similar elements when disclosing the various embodiments. It should also be appreciated that features disclosed with respect to one embodiment may be applied to other embodiments where such features would be considered appropriate for specific purposes.

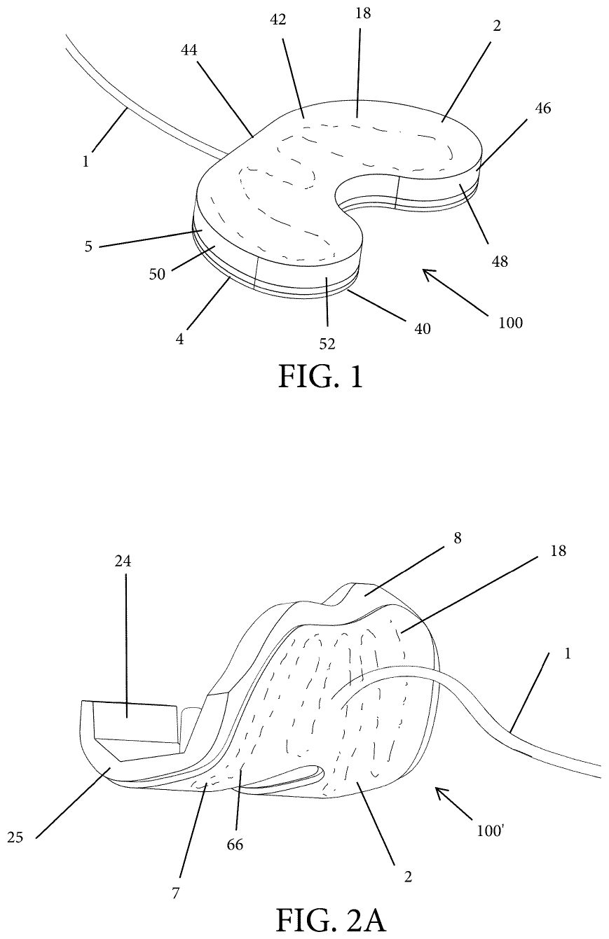

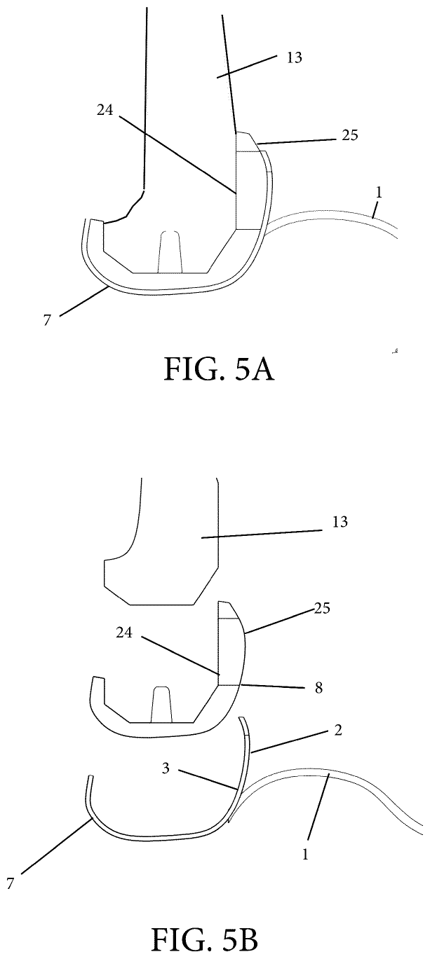

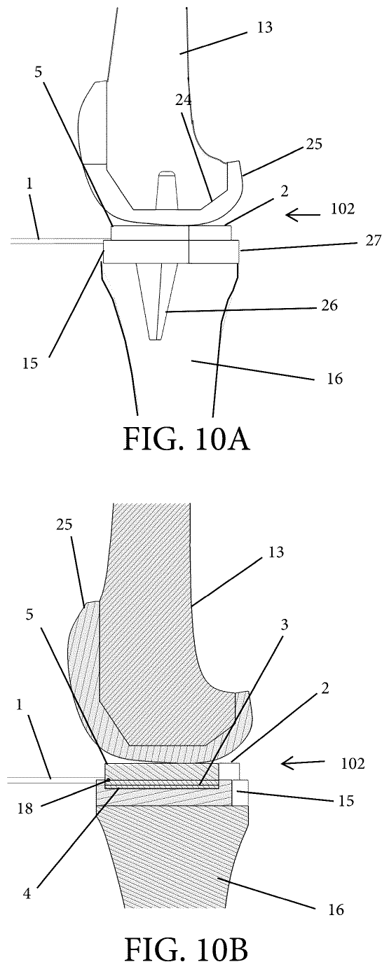

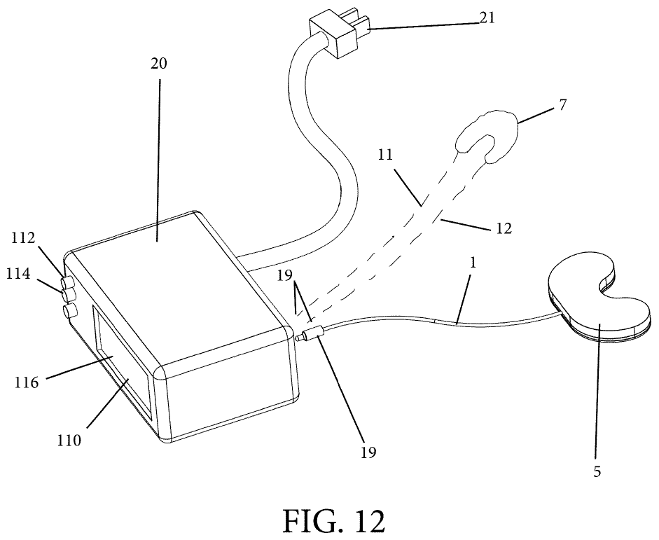

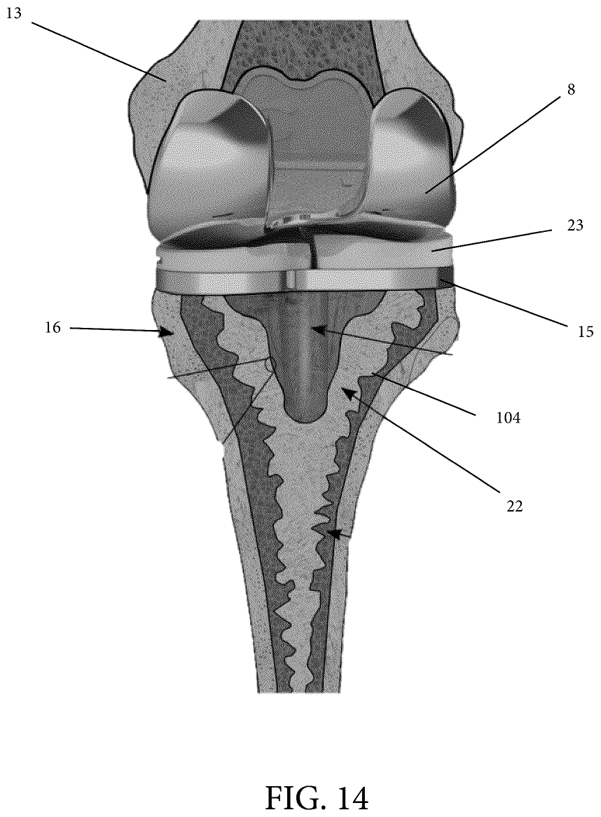

Referring to the FIGS. 1-14, various embodiments of a versatile resistance heating device 100, 100′ (for example, with 100 used to refer to the heating device embodiment specifically adapted for a tibial component 27 of an orthopedic implant and 100′ used to refer to the heating device component specifically adapted for a femoral component 25 of an orthopedic implant) designed to facilitate various orthopedic surgical procedures are disclosed. The heating device 100, 100′ comprises a heating element 18 encapsulated in a biocompatible casing 5, 7. The heating device 100, 100′ is used in conjunction with a control unit 20 (see FIG. 12), with precise temperature and duration settings, providing precise and reliable operation of a heating element 18 of the heating device 100, 100′ by managing power delivery, regulating temperature, and ensuring safe and effective functioning during orthopedic surgical procedures. An attachment mechanism 4 is further provided for securing the heating device 100, 100′ to the implant 102.

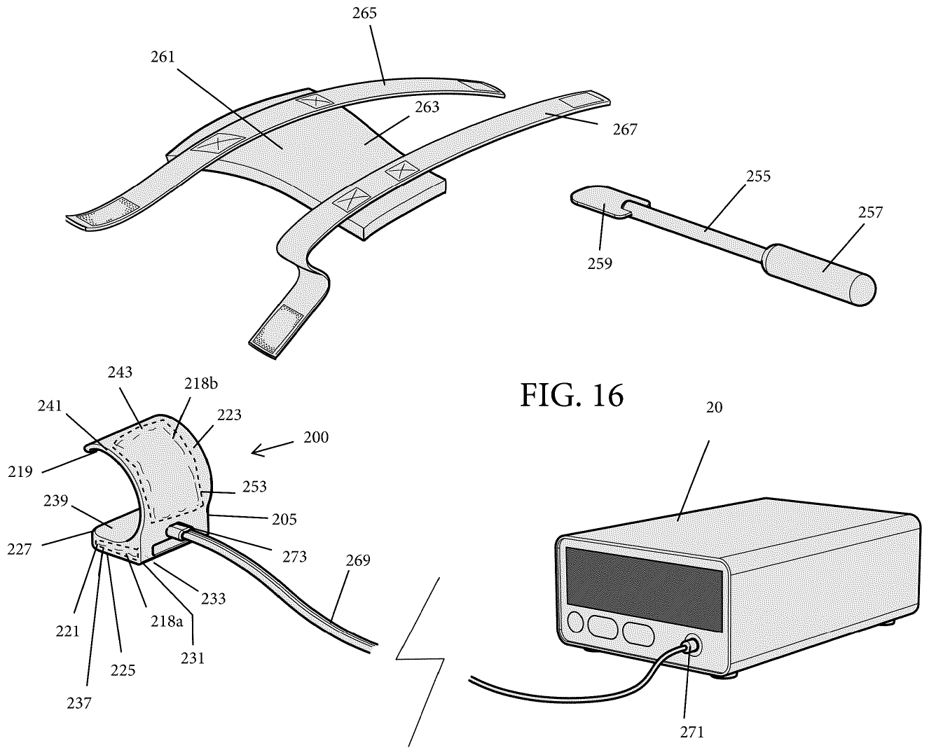

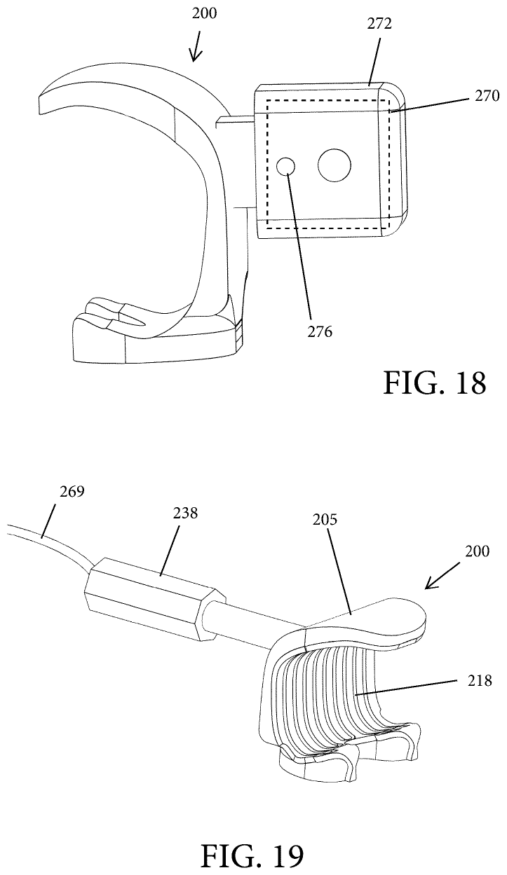

The heating device 100, 100′ operates by applying controlled heat directly to the exposed surfaces 8, 15 of an implant 102. This heat not only affects the exposed surfaces, but it can also diffuse throughout the implant 102 into the non-exposed surfaces 24, 26, thereby heating the entire implant 102. The heating element 18 is powered by either direct wall power 21 (see FIG. 12) or a rechargeable battery 270 (see FIG. 18, showing a heating device 200 similar to that disclosed with reference to FIG. 16 that includes a battery attachment 272 on the back of the heating device 200, wherein the battery attachment 272 can also act as a handle for the surgeon as they're positioning the heating device 200. The battery 270 can be removable and rechargeable, and the battery attachment has the buttons 276 to control the functionality of the device (red→off, green→on, for example)), providing flexibility in different surgical settings. The control unit 20 allows surgeons to set a specific target temperature or run pre-programmed cycles. Real-time temperature feedback from temperature sensors 17 ensures precise and safe operation.

The primary purpose of this heating device 100, 100′ is to enhance orthopedic surgical procedures in three key areas: loosening well-fixated implants during revision surgeries, disrupting bacterial biofilms and/or killing bacteria on infected implants, and accelerating the curing process of bone cement.

For loosening well-fixated implants during revision surgeries, the heating device 100, 100′ applies heat to the implant 102, causing the surrounding bone cement 22 to reach its glass transition temperature or a temperature at which the bone cement 22 begins to transition to a more flexible state. At this temperature, the bone cement 22 softens, weakening its mechanical properties and reducing its hold on the implant 102 and/or bone 104. In non-cemented implants, the thermal heat of the heating device 100, 100′ disrupts the bone-implant interface by causing different rates of thermal expansion and contraction between the implant 102 and the surrounding bone 104. This differential thermal expansion can break the bond at the interface, facilitating easier implant removal. Additionally, the heat can intentionally cause minor thermal necrosis of the bone 104 at the interface, further loosening the implant 102. This approach significantly shortens the duration of revision surgeries and enhances patient recovery. Additionally, more thorough implant loosening prior to removal would reduce the risk of excessive bone damage.

In the case of clearing bacteria and disrupting associated biofilm on infected implants, the heating device 100, 100′ targets heat to the affected implant 102. Such heating of the implant 102 disrupts the biofilm and is bactericidal. Acute infections are often localized to the exposed surfaces 8, 15 of the implant 102 that are accessible during revision surgery. Chronic infections may extend deeper to include the non-exposed surfaces 24, 26 as well. The heating device 100, 100′ targets both types of infections either by varying the shape of the heating element 18, temperature, duration, or any combination thereof. Higher temperatures and prolonged durations may be used to treat chronic, deeply embedded biofilms, whereas shorter, lower-temperature cycles may suffice for acute, surface-level infections. This level of customization allows clinicians to effectively address a broad range of infectious scenarios using a single platform. This method increases the effectiveness of traditional infection control measures, such as antibiotics or cleaning and surgical debridement methods. By enhancing the disruption of biofilms and reducing bacterial burden, the heating device 100, 100′ improves the overall success rate of infection treatments and reduces the need for additional surgeries, including implant removal. This approach contributes to better patient outcomes, lowers the risk of persistent or recurring infections, and reduces the need for further morbid surgeries.

For accelerating the curing process of bone cement 22, the heating device 100, 100′ provides controlled heat to the implant 102, which raises the temperature of the cement 22, accelerating its hardening process. Elevated local temperatures have been proven to accelerate the curing of bone cement 22. Reducing cement cure time directly correlates with reduced surgical time and, therefore, less time under anesthesia for patients. Faster curing times while implants are held in correct alignment lead to more stable implant fixation, improved implant alignment, and increased surgical efficiency, contributing to better overall surgical outcomes.

Before proceeding with a more detailed disclosure of the heating device 100, 100′ a general overview regarding electric resistance heating and implants is provided.

### Electric Resistance Heating

This heating device 100, 100′ utilizes heating technology to provide controlled and precise application of heat to orthopedic implants. The primary method employed for heating in the heating device is electric resistance heating.

Electric resistance heating operates on the principle of Joule heating, where electrical energy is converted into heat energy by passing an electric current through a resistive material. When current flows through the resistive heating element, the inherent resistance of the material to the flow of electrons causes it to produce heat. The amount of heat generated can be precisely controlled by adjusting the voltage, current supplied to the heating element, or the material or dimensions of the heating element.

### Implants

Orthopedic implants are medical devices surgically inserted into the body to replace or support damaged bones and joints. These implants are typically composed of biocompatible materials that possess excellent mechanical properties. The materials used for these implants include metals and metal alloys such as titanium, cobalt-chromium alloy, stainless steel, zirconium, nickel, and combinations thereof. Other non-metallic materials, such as ceramics and polymers, are known to be used and also benefit from the heat imparted by the present heating device. These materials are chosen for their strength, durability, resistance to corrosion, and biocompatibility, ensuring they perform well within the human body over extended periods. As disclosed herein the term “implant” is used to generally disclose the broad class of orthopedic medical devices inserted into the body to replace or support damaged bones and joints (and implants are generally referenced by the numeral 102). Where appropriate, specific implants, for example, tibial, femoral, etc., are disclosed (and include specific reference numerals specific to the specific implant).

### Materials

The materials used in orthopedic implants are often thermally conductive, which is a crucial property for the heating process facilitated by this invention for implant removal, curing bone cement, and treating infections. Examples of suitable conductive materials include, but are not limited to, nickel, silver, copper, gold, aluminum, tungsten, iron, platinum, tin, titanium, cobalt-chromium alloy, stainless steel, zirconium, and combinations thereof. These materials are characterized by their specific electrical and thermal properties, such as resistivity and conductivity, which determine the amount of heat that can be effectively delivered and managed within the implant during surgical procedures. For treating infections on exposed implant surfaces, the implants do not require thermal conduction properties. While these implants may be conductive, they may also include or be made entirely from non-conductive or less conductive components, such as composites, polymers, or ceramics. Implants consisting of multiple differing materials would be applicable as well such as a metallic implant with a ceramic coating.

Location within the Body

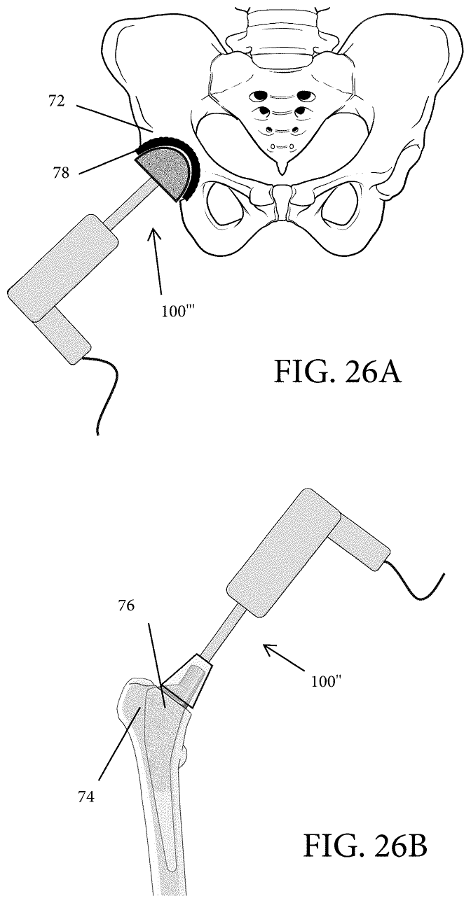

Orthopedic implants are used in various locations within the body, primarily in major joints and around bones that are susceptible to wear, damage, or disease. Common sites for these implants include the knee 13, 16 and hip joints 72, 74 where joint replacement procedures are performed. Joint replacement can also be performed in other joints, including the ankle, shoulder, and elbow. Although the majority of the disclosure focuses upon the heating device in use with knee implants, FIGS. 26A & 26B show a heating device 100″, 100″′ in accordance with the present invention used in conjunction with a hip implant, specifically, in conjunction with the femoral component 76 and the acetabulum components 78.

Considering now the knee implant, Components of knee replacement prostheses include femoral components (that is, the femoral side of knee implants) 25, tibial components (that is, the tibial side of knee implants) 27, and sometimes patellar components. In the hip joint, hip replacement implants often comprise components such as the femoral stem, the acetabular cup, and the femoral ball, which are inserted into and around the femur and the acetabulum of the pelvis. For the shoulder joint, implants may include the humeral component and the glenoid component, which are placed in and around the humerus and the scapula. Elbow implants typically replace parts of the distal humerus and proximal ulna and may consist of components that articulate to replicate the natural joint motion. Ankle implants typically replace portions of the distal tibia and articular talus.

### Components

Orthopedic implants can be either unitary devices or composed of multiple components, depending on the surgical requirements and the specific joint being treated. Unitary devices consist of a single component designed to replace or support a specific part of the joint, such as a hip hemi-arthroplasty, where there is only a femoral component. Multi-component devices consist of two or more components that work together to restore joint function. For example, a total hip replacement may consist of a femoral and a separate acetabular component. A total knee component may consist of a femoral component 25, tibial component 27, and patellar component. In some embodiments, the heating device is designed to heat multiple components simultaneously (see the embodiment of FIG. 26). For instance, in a knee replacement prosthesis, the heating device 200 may be used to heat both the femoral component 25 and tibial component 27 simultaneously. In other embodiments, the heating device 100, 100′ is used to selectively heat a single component. For instance, in a knee replacement prosthesis, the heating device 100, 100′ may be used to selectively heat the femoral component 25 but not the tibial component 27 or vice versa.

This selective heating is based on the surgical goal, such as loosening the implant 102, accelerating cement curing, or aiding in clearing infection. Similar applications of heating multiple or single components can be applied to hip replacement prostheses as well.

### Exposed Surfaces

The exposed surfaces of an orthopedic implant 102 are the areas that a surgeon can directly access after performing a surgical exposure in the patient. The exposed surfaces are usually accessible for manipulation or application of external devices. Additionally, the articular surfaces of joint replacement implants are considered as exposed surfaces. For instance, in a knee replacement surgery, the exposed surfaces might include the superior surface 15 of the tibial component 27 or the femoral articular surface 8 of the femoral component 25. These are the areas that the heating device 100, 100′ will contact to apply targeted thermal energy.

Exposed surfaces can be part of single or multi-component implants. In a multi-component implant 102, the exposed surfaces of one component might contact the exposed surfaces of another. For example, in a total knee replacement, the femoral component 25 and the tibial component 27 have exposed surfaces 8, 15 that interact with each other or with an intermediary liner 23. These surfaces are critical for joint movement and functionality and are accessible during surgery for adjustments or interventions.

In the case of infections, the heating element is directly applied to the exposed surfaces to aid in biofilm and bacterial disruption. In this configuration, the heating device 100, 100′ delivers localized thermal energy that raises the temperature of the implant surface in direct contact with the biofilm or the biofilm directly. This thermal exposure denatures bacterial proteins, disrupts the extracellular polymeric matrix, and significantly impairs the structural integrity of the biofilm, thereby facilitating mechanical removal and improving the efficacy of antibiotic therapies. Additionally, the increase in implant surface temperature can be bactericidal.

### Non-Exposed Surfaces

Non-exposed surfaces, on the other hand, are the parts of the implant 102 that remain in direct contact with cement 22, bone 104, are embedded within the bone structure, or are not easily accessible through a routine surgical approach. These surfaces include any part of the implant 102 that is directly fixated within the bone canal, such as the stem 24 of a hip implant or the stem (or keel) 26 of a knee implant. Non-exposed surfaces might be bonded to the bone 104 using bone cement 22 or through a cementless fixation process, where new bone growth integrates with the implant surface. Such surfaces are not accessible for direct application of the heating device 100, 100′ due to their position within the bone 104 or their continuous contact with the bone tissue. Non-exposed surfaces can be accessed if the surgeon elects to modify the bone or manipulate the implant 102 to gain access to the non-exposed surfaces of the implant 102.

For cemented implants, non-exposed surfaces 24, 26 are typically surrounded by a layer of bone cement 22, which acts as a filler and locking agent between the implant 102 and the bone 104. This cement 22 extrudes into the microscopic structure of the bone 104, creating a stable and durable bond. For cementless implants, these non-exposed surfaces 24, 26 often have a textured or porous coating that encourages new bone growth, securing the implant 102 over time through biological integration. The heating device's role in these contexts is to target the accessible exposed surfaces 8, 15, indirectly affecting the non-exposed surfaces 24, 26 by thermal conduction.

For chronic infections, the bacterial burden and/or biofilm can extend beyond the exposed surfaces and colonize non-exposed regions of the implant 102 that are in contact with bone 104 or encapsulated in bone cement 22. Although these regions are not directly accessible, the heating device 100, 100′ still contributes to their treatment. By applying sustained heat to the accessible exposed surfaces, thermal conduction through the implant body raises the temperature of adjacent non-exposed surfaces. This indirect heating effect, dependent on the duration, temperature settings, and thermal properties of the implant 102, enables the disruption of bacteria and/or biofilms residing in deeper, less accessible areas. The effectiveness of this approach is tailored by selecting appropriate heating durations, temperatures, and device geometries to ensure adequate thermal diffusion, making the heating device 100, 100′ suitable for treating both acute and chronic infections.

In many cases, modular parts of the implant 102 can be removed to gain better access to the exposed surfaces 8, 15. For example, in a knee replacement implant, the polymer liner 23 may be removed to expose the underlying superior surfaces 15 of the tibial implant 27 and femoral articular surface 8 of the femoral component 25. This removal provides a direct path for the heating device 100 to apply targeted thermal energy to the metallic components, enhancing the effectiveness of the procedure. The heating device 100, 100′ can also apply heat to these modular components if they have become infected and the surgeon elects to reuse them.

The heating device 100, 100′ and its components are now described in more detail. Generally, the heating device 100, 100′ includes a casing 5, 7 in which a heating element 18 is positioned. The heating device 100, 100′ further includes a control unit 20 connected to the casing 5, 7 and/or the heating element 18 to apply and control the power needed to generate a desired level of heat. Temperature sensor(s) 17 are also used in conjunction with the heating element and the control unit 20 to ensure optimal heat application. It should be appreciated that the following disclosure focuses upon the use of the present heating device 100, 100′ in conjunction with the tibial side of knee implants and the femoral side of knee implants. However, it is appreciated that the concepts disclosed herein are readily applicable to uses in conjunction with a variety of implants.

### Heating Element

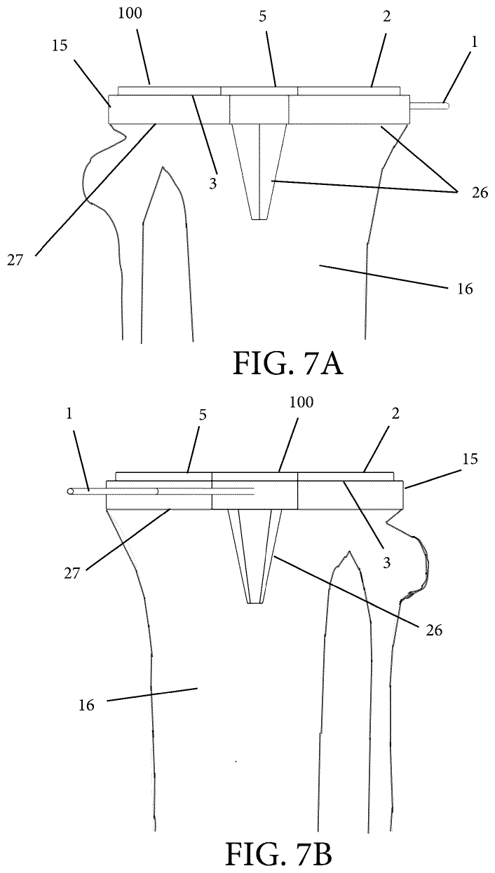

Referring to the embodiment disclosed with reference to FIGS. 7A-B, 8A-B, 9A-B, and 10A-B the heating element 18 is the core component of the heating device 100, designed to deliver controlled and uniform heat to implants 102. As discussed below in detail, the heating element 18 is made from a high-resistivity alloy, allowing the heating element 18 to efficiently convert electrical energy into thermal energy. This heating element 18 comprises a resistance heating wire 106 arranged in specific shapes or patterns, such as serpentine or spiral configurations, to ensure optimal heat distribution across the implant surface. The heating element 18 can be configured in a geometrical pattern that allows it to bend, contour, expand, and contact without causing damage. The robust construction of the heating element 18 guarantees reliable performance and durability throughout surgical procedures. While the heating element is only shown in these figures, it is appreciated the heating element forms a part of each of the embodiments disclosed herein.

In practice, and as disclosed below in accordance with the various embodiments, the heat may be applied so that it diffuses to the non-exposed surfaces of the implant, so that it is localized to the exposed surface. Further still, the heating device can be used to heat implants prior to implantation and to heat the implant while the implant is in situ.

### Material

The heating element 18 is a crucial component of the invention. It is designed to provide consistent and controlled heat to the targeted areas of implants. The materials selected for the heating element 18 are essential for ensuring its efficiency, biocompatibility, and durability in surgical environments.

One example of a material that can be used for the heating element 18 is Nichrome wire, an alloy consisting primarily of nickel and chromium. Nichrome is known for its high electrical resistivity and excellent resistance to oxidation at high temperatures, making it an ideal choice for resistance heating applications. When an electric current passes through Nichrome wire, its resistive properties generate heat uniformly, providing a reliable source of thermal energy.

Another potential material for the heating element 18 is KANTHAL™, an iron-chromium-aluminum alloy. KANTHAL™ is valued for its ability to operate at even higher temperatures than Nichrome and its superior oxidation resistance. This material's high melting point and stability make it suitable for applications requiring prolonged heating cycles. When used as a heating element 18, KANTHAL™ wire heats quickly and maintains consistent temperatures, ensuring effective thermal treatment of orthopedic implants. While Nichrome and KANTHAL™ are disclosed herein, the heating element 18 can also be constructed from other resistive materials or composite conductors that offer desirable electrical and thermal characteristics. Future embodiments may also explore advanced resistive materials such as carbon nanotube-infused composites or graphene-based films, offering potential advantages in miniaturization and thermal performance, provided that biocompatibility and thermal stability are verified.

Resistance heating relies on the intrinsic properties of the material used. When a voltage is applied across the wire, the material's resistance to electric current causes it to generate heat electrical energy that is converted to thermal energy. This process is governed by Joule's Law, which states that the heat produced (H) is proportional to the square of the current (I), the resistance (R) of the material, and the time (t) for which the current flows: H=I²Rt. By carefully selecting the resistance properties of the wire and controlling the current, the heating device 100, 100′ can achieve precise temperature regulation.

In operation, the heating element 18 exploits the resistive heating principle, where the chosen material's high resistivity ensures efficient heat generation. The material's thermal stability allows it to reach and maintain the required temperatures without undergoing structural or chemical changes, ensuring consistent performance throughout the procedure.

The heating element's design also considers the need for rapid and uniform heat distribution. For instance, Nichrome's and KANTHAL™'s properties enable quick heating upon the application of current, ensuring that the target temperature is reached swiftly and maintained consistently. This rapid response is critical in surgical settings, where time efficiency and precision are crucial.

Additionally, depending on the surgical application, the heating element 18 can be fabricated from flexible or malleable materials that enable it to bend and conform to the surface geometry of different implant types. This allows the heating device 100, 100′ to accommodate both curved and flat implants. Conversely, if the heating device 100 is to be rigid and non-flexible, then the heating element 18 can be constructed from a more rigid material as shown with reference to the embodiment disclosed in FIGS. 7A-B, 8A-B, 9A-B, and 10A-B.

If the heating element 18 is directly exposed to the implant 102, it may be constructed from biocompatible materials, such as titanium alloys, tantalum, platinum and platinum alloys, gold, and other conductive polymers that are sterilizable (see the embodiment disclosed with reference to FIG. 19 that shows the heating element 218 is exposed and not embedded within the casing of the heating device 200. This allows the heating element to directly contact the surface of the implant 102.

Conversely, if the heating element 18 is encapsulated within a biocompatible housing (see the embodiment of FIGS. 7A-B, 8A-B, 9A-B, and 10A-B), then the biocompatibility and sterility of the heating element 18 are less of a concern.

### Size/Shape

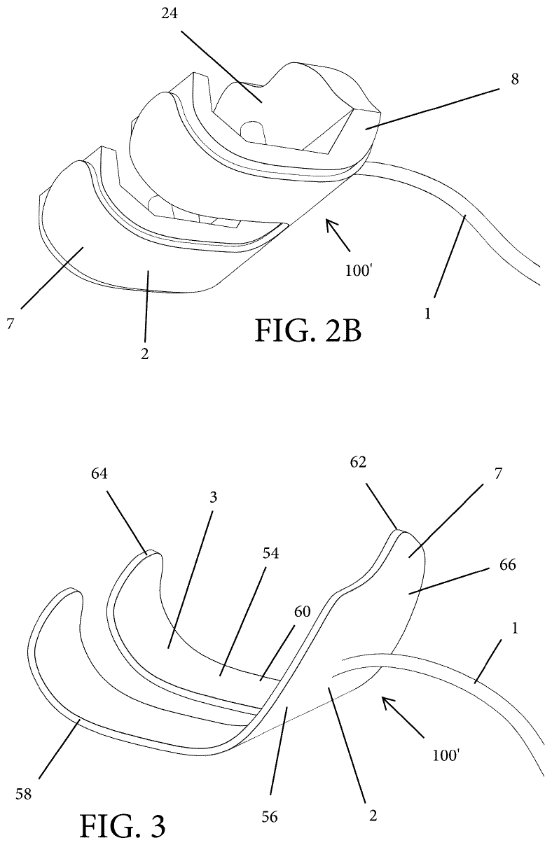

The heating element 18 is designed to match the specific contours and dimensions of the targeted implant's exposed surfaces. For example, and with reference to FIGS. 2A, 2B, 3, 4A, 4B, 5A, 5B, 6A, 6B, 6C, 11A, flexible heating elements can be designed for curved surfaces such as the femoral component 25 of knee implants, ensuring full contact and uniform heat distribution. Conversely, rigid heating elements may be more suited for flat surfaces, like the tibial component 27 of knee implants, providing a stable and consistent heating profile (see FIGS. 1, 7A-B, 8A-B, 9A-B, 10A-B, 11B). Rigid heating elements can also be used for curved surfaces, as long as the heating element 18 is manufactured in a preformed manner that allows it to contour to the curved surface. The heating element may have a shape memory attribute, allowing it to contour to varying geometries. Customizability in size and shape allows the heating element 18 to be adapted for use with different types of implants, including those used in the hip, ankle, shoulder, and elbow.

The pattern of the resistance wire 106 within the heating element 18 is another crucial aspect. The wire 106 can be arranged in various configurations, such as serpentine or spiral patterns, to ensure even heat distribution across the implant surface. A serpentine pattern, for example, maximizes the surface area of the wire in contact with the implant 102, promoting uniform heating and preventing hotspots that could damage surrounding tissues or compromise the integrity of the implant 102. The precise arrangement of the resistance wire 106 is designed to achieve a balanced heating profile, ensuring that the entire surface of the implant 102 is heated uniformly and efficiently. Some heating element configurations may include a higher wire density in one or more certain areas to correspond with thicker regions of the implant to promote uniform implant heating.

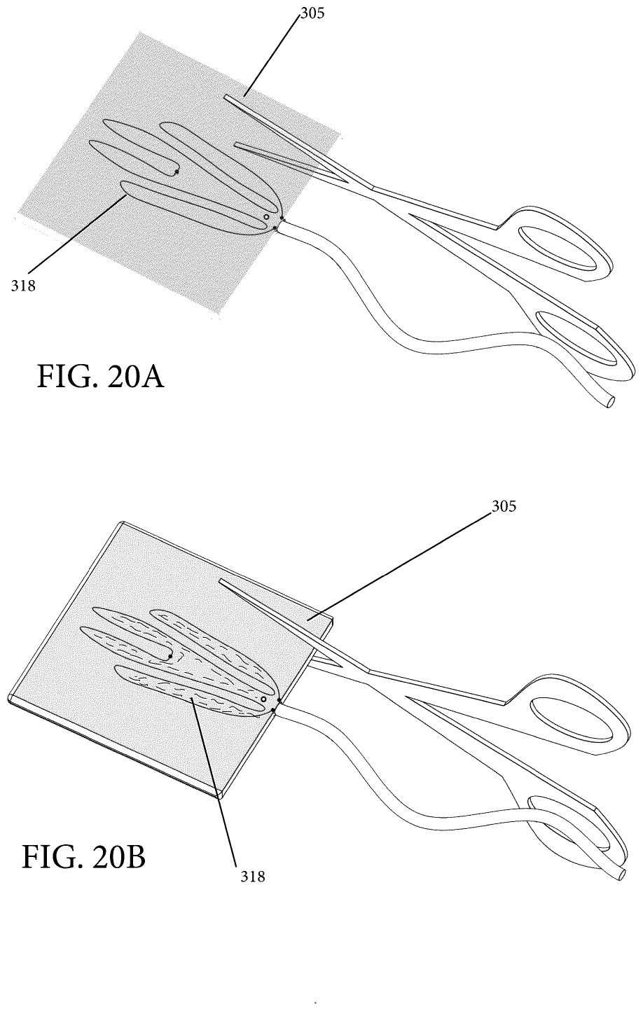

In accordance with an alternate embodiment, the heating element 318 can also be manufactured in a way that enables the surgeon to modify or cut the casing 305 to better fit the implant geometry (as discussed below with reference to FIGS. 20A & 20B). Similarly, if the surgeon is unable to contour the heating element 18 to contact the entire implant surface, fillers and thermal pastes can be used to bridge the gap and allow heat to be conducted from the heating element 18 to the implant 102.

Additionally, and in accordance with a disclosed embodiment, the heating element 18 can be designed to accommodate real-time temperature feedback mechanisms. Temperature sensors 17, such as thermistors or thermocouples, can be strategically placed within or around the heating element 18 to monitor and regulate the temperature accurately. This integration ensures that the heating device 100, 100′ operates within safe and effective temperature ranges, preventing overheating and enhancing the precision of the thermal treatment.

### Temperature

The temperature range of the heating element 18 is a critical parameter that directly influences its effectiveness in various surgical applications. One objective is to heat the implant 102 to reduce the mechanical properties of the bone cement 22, which typically ranges from 60° C. to 140° C. This temperature range is crucial for softening the bone cement 22, thereby facilitating the removal of well-fixed implants.

To achieve the desired temperature at the bone-implant, bone-cement or implant-cement interface, the heating element 18 itself may need to reach higher temperatures. This is because the heat must penetrate through the implant material and any intervening layers to effectively raise the temperature of the bone cement 22 to its softened state. Depending on the thermal conductivity of the implant material and the thickness of the implant 102, the heating element 18 may need to operate at temperatures significantly above 140° C. For instance, if the implant 102 is made of stainless steel or titanium, known for their high thermal conductivity, the heating element 18 might need to reach temperatures of 100° C. to 150° C. to ensure adequate heat transfer. Additionally, this application can be utilized with non-cemented implants. The heat can disrupt the bone-implant interface at an appropriate temperature.

In addition to achieving the glass transition temperature of the bone cement 22, the heating element 18 must also be effective in other surgical scenarios. For example, in treating infections, the heating element 18 should reach temperatures sufficient to be bactericidal and/or disrupt the biofilm matrix, typically around 60° C. to 100° C.

For accelerating the curing process of bone cement 22, the heating element 18 must provide a controlled and consistent temperature to enhance the exothermic polymerization reaction of the cement 22. Typically, bone cement polymerizes faster at elevated temperatures, with optimal curing temperatures ranging from 40° C. to 100° C. By maintaining the bone cement 22 at these temperatures, the device can significantly reduce the time required for the cement 22 to harden, thus shortening the overall surgical procedure.

### Casing

The heating element 18 is supported by a casing. As such, the heating element may be embedded or encapsulated within the casing or the heating element may be positioned along an external surface of the casing so that it can be directly exposed to the implant surface.

The heating element 18 is housed within a casing, for example a casing 5 for a tibial component 27 (see FIGS. 1, 7A-B, 8A-B, 9A-B, 10A-B, 11B) or a casing 7 for a femoral component 25 (see FIGS. 2A, 2B, 3, 4A, 4B, 5A, 5B, 6A, 6B, 6C, 11A). The casing 5, 7 for the heating element 18 is an essential component of the invention, designed to house and protect the heating element 18 while ensuring patient safety. Its primary purpose is to support the heating element 18 and shield it from mechanical damage and environmental contaminants. Additionally, the casing 5, 7 prevents direct exposure of the patient to the heating element 18, protecting surrounding tissues from potential burns or thermal injury. Constructed from durable, biocompatible materials, the casing 5, 7 can withstand high temperatures and repeated sterilization cycles, ensuring both the longevity and sterility of the device.

As will be appreciated based upon the various embodiments disclosed herein, the casing is manufactured in a shape that has a similar geometry and shape as the portion of the implant component it is intended to contact. As such, and when considering curved surfaces of implants, the casing will have a similar or slightly dissimilar radius to the general radius of the implant, for example, a femoral implant. Further, the casing may be constructed from a variety of materials but is ultimately constructed from materials that allow it to conform and hold its shape relative to the implant, while maximizing contact with the exposed implant surface.

With this in mind, the casing may be manufactured from rigid materials allowing it to interface directly with the implant shape. The casing may also be made from malleable materials allowing it to be intraoperatively molded to the implant shape, wherein the casing is flexible for easily conforming to the implant surface geometry. The casing can be a uniform material or made from a combination of materials of differing mechanical and/or conductive properties. In addition, the casing may be manufactured in a way that allows it to be trimmed or modified to the desired shape which will not disrupt or damage the heating element.

One approach to encasing the heating element involves first forming a base layer of casing material, either as a pre-molded solid or a cut-to-shape sheet. The heating element 18 is then placed on top of this base layer and arranged into its desired configuration (e.g., serpentine, spiral, or looped). A top casing layer is then positioned over the heating element and bonded to the bottom layer using a medical-grade adhesive, heat, and/or pressure. This method seals both layers together and creates a sealed and durable enclosure around the heating element, maintaining flexibility (if needed) and ensuring consistent positioning of the element during operation.

In another embodiment, the casing is formed using a liquid or gel material (such as silicone rubber or medical-grade polyurethane) that is poured into a mold to create the bottom layer. As this material begins to cure, the heating element 18 is embedded into the partially cured base, allowing it to become integrated with the structure. Once the base layer is fully or nearly cured, a top layer of the same or a different compatible material can be either poured or placed over the heating element and bonded to the bottom layer. This method results in permanent encapsulation of the heating element and can provide a seamless or near-seamless interface that minimizes edges or gaps.

### Material

The materials selected for the casing 5, 7 of the heating element 18 are crucial for ensuring both the device's performance and patient safety. The casing 5, 7 must be constructed from biocompatible materials to prevent any adverse reactions when in contact with bodily tissues. These materials must also be sterilizable to maintain the necessary sterility standards required in surgical environments. The casing 5, 7 may also be designed to withstand moderate mechanical stress or compression during surgical manipulation, ensuring the heating element 18 remains protected during routine or accidental contact with surgical instruments or when it is compressed against an implant surface. The casing materials are selected or layered to provide sufficient electrical insulation, minimizing the risk of current leakage or electrical interaction with surrounding surgical tools or tissues.

Heat resistance is another essential property of the casing materials. The casing 5, 7 must withstand the high temperatures generated by the heating element 18 without degrading or losing structural integrity. Materials such as, but not limited to, medical-grade silicone rubber, polyimide film, PTFE (polytetrafluoroethylene), stainless steel, aluminum, or medical-grade PEEK (polyether ether ketone) are potential candidates, as they can endure prolonged exposure to elevated temperatures.

The flexibility or rigidity of the casing 5, 7 depends on the specific application. For instance, and in accordance with one embodiment, the casing 5, 7 comprises flexible materials like silicone rubber, polyimide film, or PTFE for use when the heating element 18 needs to conform to curved implant surfaces, such as the femoral side of knee implants 25. These materials offer the advantage of being pliable yet durable, ensuring that the casing 5, 7 can adapt to various shapes without compromising its protective function.

On the other hand, and in accordance with another embodiment, the casing 5, 7 comprises rigid materials like stainless steel, aluminum, or medical-grade PEEK when the heating device 100 is intended for applications involving flat or structurally stable surfaces, such as the tibial component 27 of knee implants. These materials provide robust protection for the heating element 18 and maintain their shape under stress, ensuring consistent performance.

In addition to biocompatibility, sterilizability, and heat resistance, the materials chosen for the casing 5, 7 should also be chemically inert to avoid reactions with bodily fluids or other substances encountered during surgery. This ensures that the casing 5, 7 remains safe and effective throughout its use.

The casing 5, 7 of the heating element 18 does not need to be uniform in its material properties. The first side (that is, the heating side) 3 of the casing 5, 7 that contacts the exposed surface 8, 15 of the implant 102 can be made from a thermal conductor to ensure efficient heat transfer directly into the implant 102. Materials such as medical-grade aluminum or copper can be used for this purpose, facilitating rapid and uniform heating of the implant surface. In contrast, the opposed second side (that is, the outer side) 2 of the casing 5, 7 can be constructed from a thermal insulator, such as silicone rubber or PTFE, to minimize heat loss and protect surrounding tissues from unintended thermal exposure. This multi-material approach focuses the heat generated by the heating element 18 into the implant 102, enhancing the efficiency and effectiveness of the thermal treatment while ensuring patient safety and optimizing the device's performance. Additionally, multiple materials may be utilized so that certain areas of the casing are flexible while others are rigid.

The casing 5, 7 can also be uniform if a single device is used to heat multiple implants simultaneously. In this instance, heat from the heating element 18 can diffuse in both directions towards each implant 102.

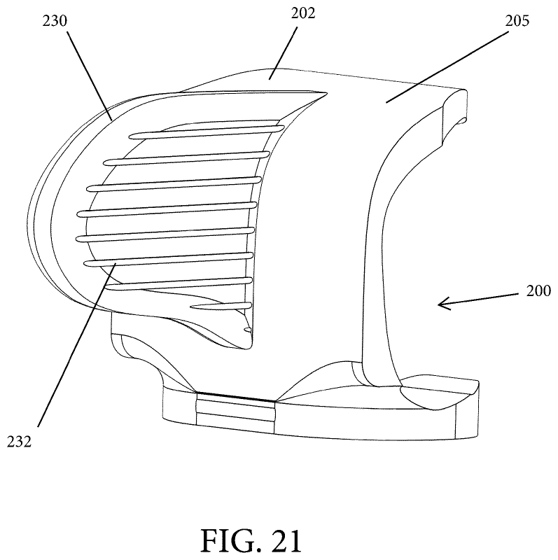

The casing 5, 7, along the heating side 3 thereof, may include a deformable, flexible or semi-solid like surface that can conform to the irregularities of the implant surface as shown in FIGS. 21A-B. For instance, the casing 5, 7 for the heating element 18 to heat a knee replacement tibial tray can conform to the tray edges, machined grooves, and locking mechanism which will improve surface contact and implant heating.

To aid with surgical handling, the outer surface of the non-heating side of the casing may be textured or coated with a non-slip, biocompatible polymer to enhance grip and placement stability during the procedure. Such an embodiment is shown with reference to FIG. 21 wherein the opposed second side (that is, the outer side) 202 of the casing 205 is provided with a handle 230 with a grippy, non-slip texture 232.

### Size/Shape

The size and shape of the casing 5, 7 for the heating element 18 are meticulously designed to ensure optimal performance and compatibility with various implant surfaces. The casing 5, 7 must precisely mate with the exposed surfaces 8, 15 of the implant 102, providing uniform contact to ensure effective heat transfer and consistent therapeutic outcomes. If the casing 5, 7 is unable to mate entirely with the exposed surface, thermal paste or other thermal conductors can be used to fill the voids between the casing 5, 7 and the exposed surface.

For the casing 5, 7 to function effectively, it must be of an appropriate thickness to allow efficient heat transfer from the heating element 18 to the implant 102. The material thickness is carefully balanced to provide sufficient insulation and protection while enabling the necessary thermal conductivity. Too thick of a casing 5, 7 could impede heat transfer, while too thin of a casing 5, 7 might compromise structural integrity and protective functions.

In the case of solid, rigid casings 5, such as those designed for flat surfaces like the tibial component 27 of knee implants (see FIGS. 1, 7A-B, 8A-B, 9A-B, 10A-B, 11B), the casing 5 may be crafted to match the shape and size of the polymer component 23 of the tibial implant 27. This precise matching ensures that the casing 5 maximizes and maintains tibial implant 27 surface contact, and maintains effective heat distribution across the exposed surface 15. The rigid casing 5 must be designed to hold its shape under thermal and mechanical stress, providing a stable platform for the heating element 18. Rigid casings can also be used for curved surfaces if the casing has been manufactured and preformed to match the contours of the surface.

In accordance with such an embodiment, the casing is substantially rectangular in shape (with adjustments made in the shape to conform with the implant) and includes a lower wall 40, an upper wall 42, a first side wall 44, a second side wall 46, a third side wall 48, and a fourth side wall 50. The first, second, third, and fourth side walls 44, 46, 48, 50 extend between the exposed lower wall 40 and the upper wall 42. Within the casing member 5 and positioned between the exposed lower wall 40 and the upper wall 42 is a cavity 52 in which the heating element 18, as well as other functional components, are housed.

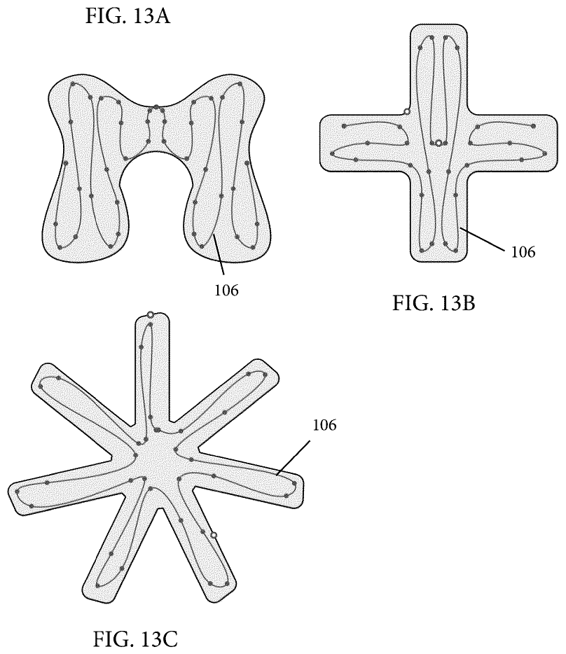

For applications requiring flexibility, such as those involving curved surfaces like the femoral component 25 of knee implants (see FIGS. 2A, 2B, 3, 4A, 4B, 5A, 5B, 6A, 6B, 6C, 11A), the casing 7 must be designed to conform to these contours without becoming damaged. Flexible materials can be employed to create a casing that adapts to the shape of the implant 102 while maintaining structural integrity. The flexible casing 7 should provide a snug fit around the femoral component 25 of knee implant, ensuring consistent heat transfer even as it flexes and adjusts to the implant's surface 8. The flexible casing 7 can also come in a variety of sizes and shapes, as shown in FIG. 13A-C, to contour to a wide variety of implant shapes and sizes. The flexible casing may have a shape memory component and slightly different curvature from the implant to better contour and increase surface area contact with the implant. For flexible casings, mesh reinforcement or layered composites that allow flex in certain zones while maintaining rigidity in others can be integrated into the casing.

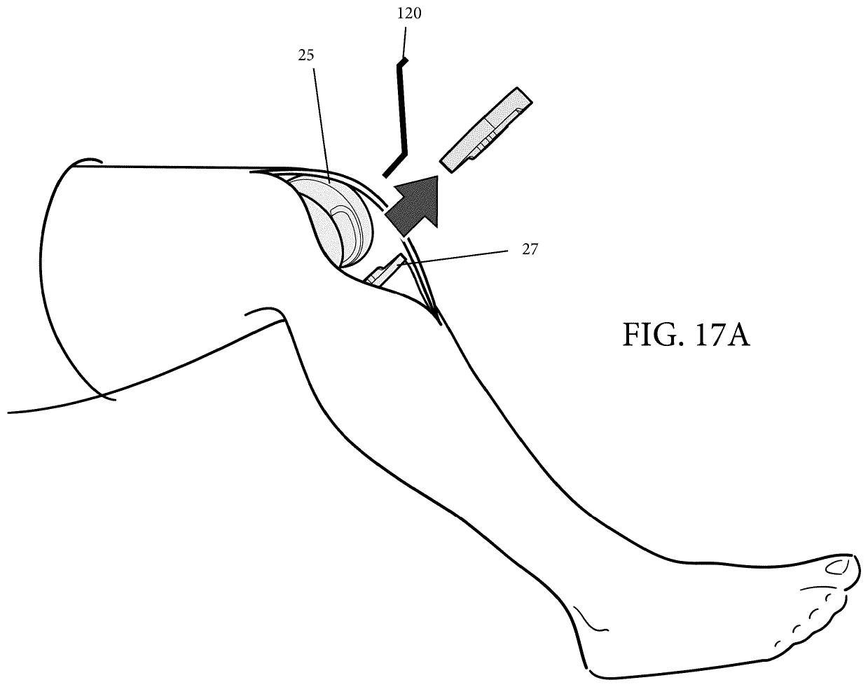

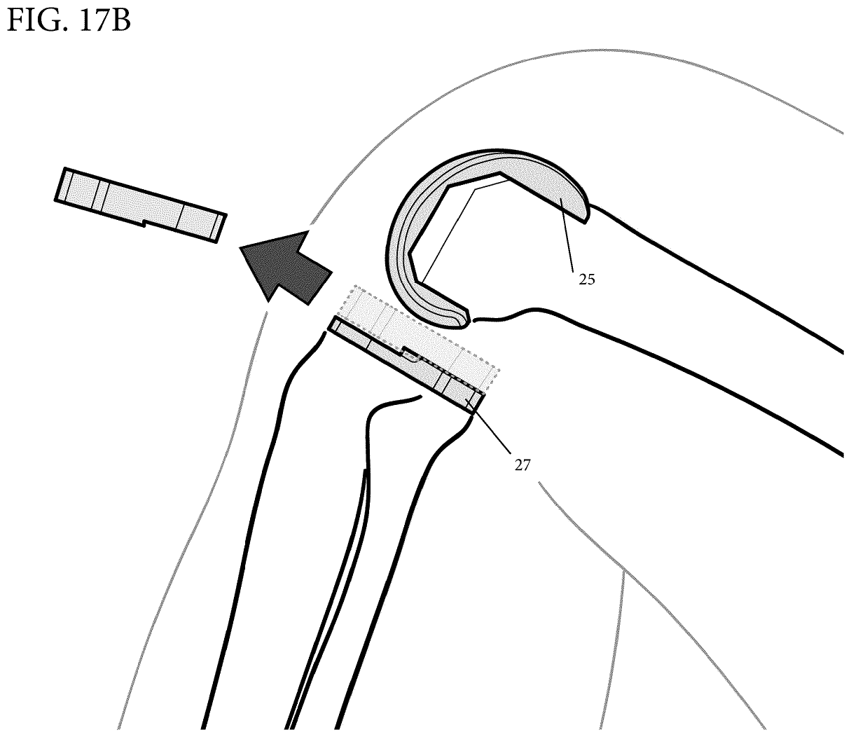

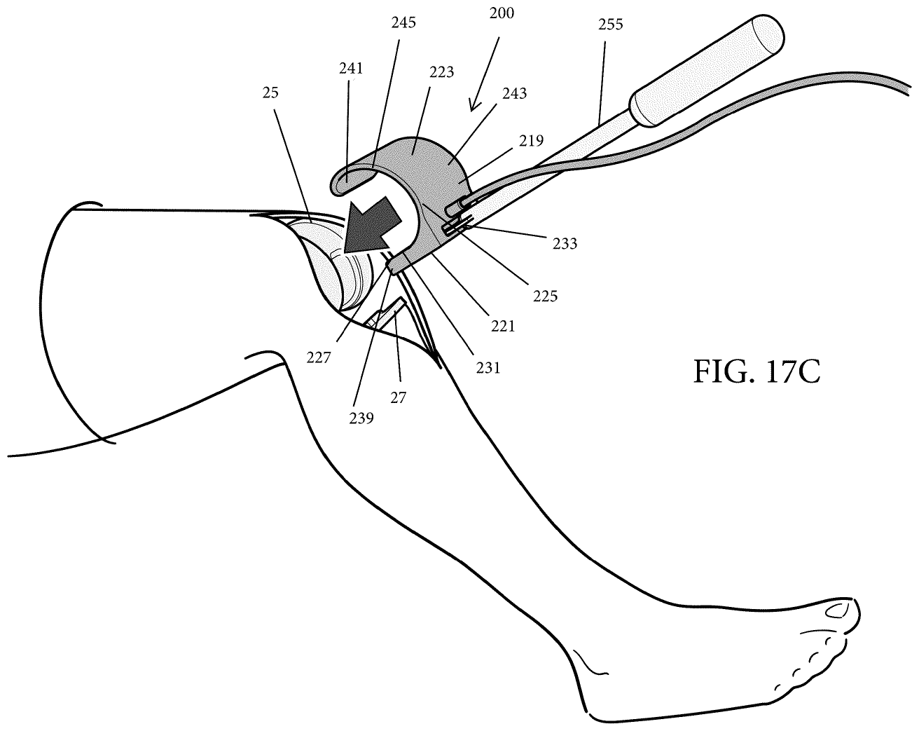

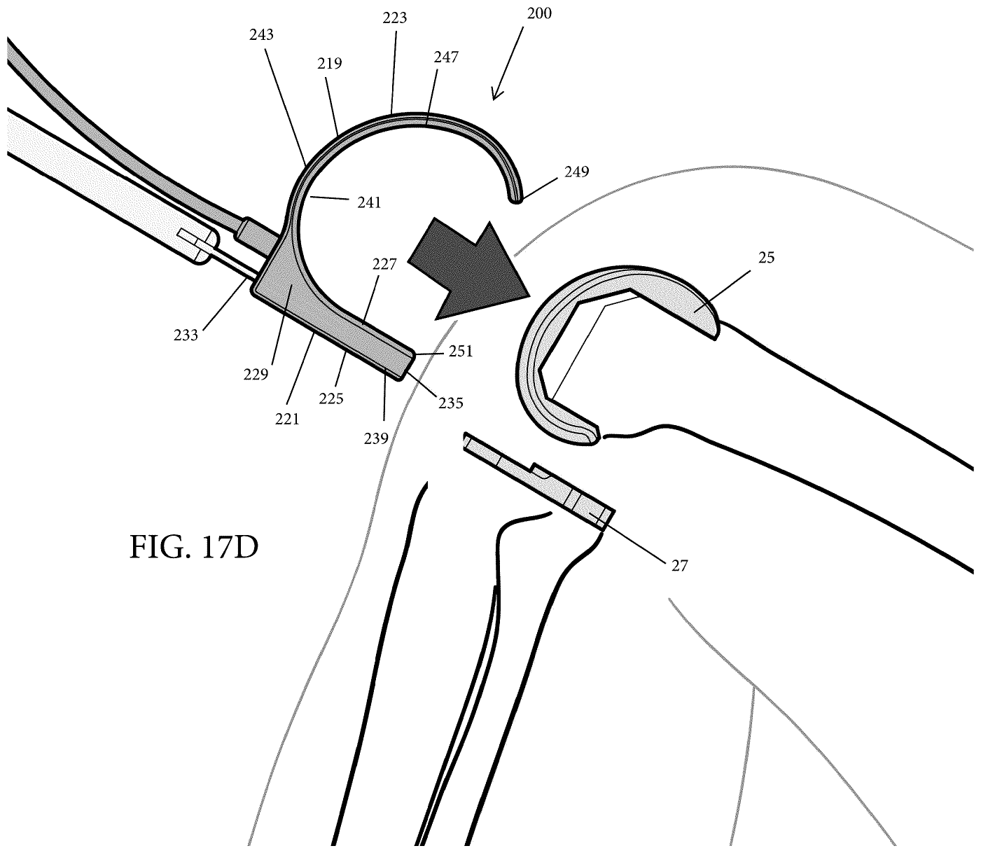

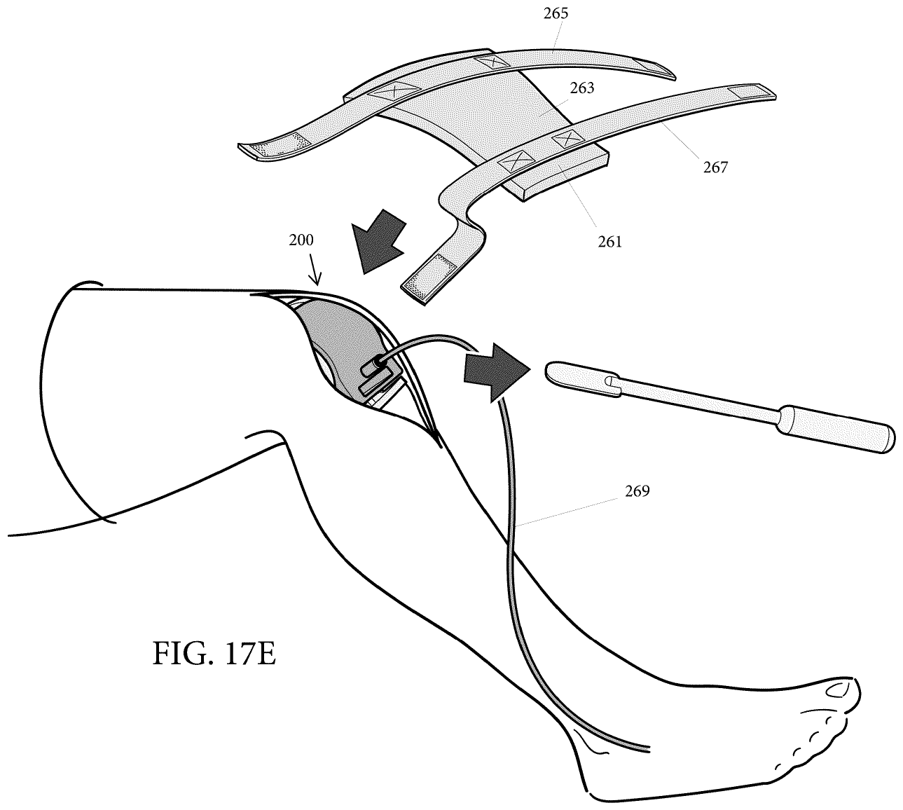

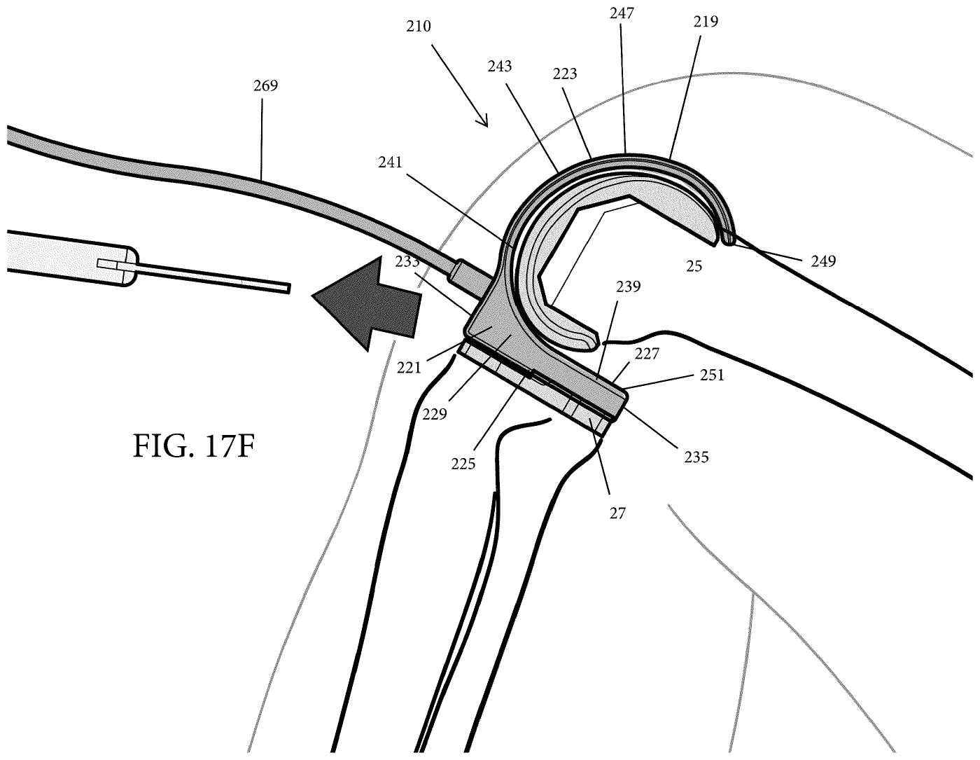

In accordance with a casing 7 structured for the femoral component, the casing 7 is substantially C-shaped and includes a concave internal wall 54 and a convex external wall 56. Extending between the concave internal wall 54 and the convex external wall 56 are a first lateral side 58, a second lateral side wall 60, a first end side wall 62, and a second end side wall 64. Within the casing 7 and positioned between the concave internal wall 54 and the convex external wall 56, is a cavity 66 in which the heating element 18, as well as other functional components, are housed. The casing 7 is sized to fit over the femoral component and is structured with resilience, allowing it to slightly open and snap over the femoral component when applied thereto in a manner described below with reference to the embodiment disclosed in FIGS. 16 and 17A-F.

The casing's design must also account for ease of attachment and detachment, ensuring that it can be securely placed on the implant during surgery and easily removed afterward. This requires consideration of the overall shape and any features that aid in secure positioning, such as grooves, tabs, or other fastening mechanisms.

Additionally, the casing 5, 7 should be designed to minimize any gaps or air pockets between it and the exposed surfaces 8, 15. These gaps can act as insulators, hindering efficient heat transfer and potentially leading to uneven heating. The shape and size of the casing 5, 7 should facilitate close, continuous contact with the implant 102, promoting optimal thermal conduction. Any gaps or air pockets can also be filled using a biocompatible thermal paste.

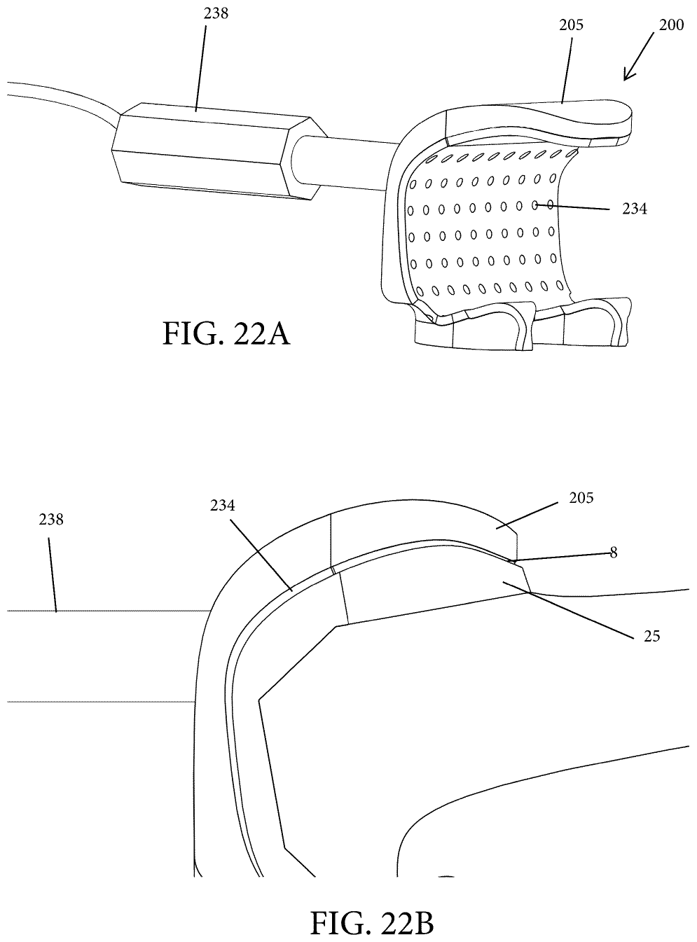

Design elements, such as tapered edges or flexible joints, can further enhance the casing's adaptability to various implant geometries. These features help ensure that the casing 5, 7 remains in firm contact with the implant surface throughout the heating process, maximizing the efficiency of heat transfer and the effectiveness of the therapeutic application. To maximize thermal coverage and efficiency, the casing 5, 7 can also be constructed with deformable or semi-flexible sections that allow it to passively conform to minor variations in implant topology, such as grooves, locking mechanisms, or ridged surfaces. This adaptability ensures consistent surface contact, improving heat transfer and minimizing thermal inefficiency. This feature is disclosed with reference to the embodiment shown in FIGS. 22A and 22B, wherein the heating device 200 has a deformable/soft/spongy layer 234 on the inner surface, which allows it to conform to the exposed surfaces 8 of the implant 25. If the casing is made from rigid materials, then the soft layer can be an addition to the contact surfaces. If the casing is made from silicone, for example, the device will inherently be soft and deformable. So the soft layer will already be a feature of that device. An additional/separate spongy layer may not be necessary. As the heating device 200 is pressed against the implant 25, it will deform so that the whole surface of the implant 25 is in contact with the heating device 2000. As shown in FIG. 22B, some areas of the deformable/soft/spongy layer 234 are compressed more than others, thus ensuring good contact if the surface of the implant 25 has irregularities in its shape, or if the surgeon doesn't 100% line up the heating device 200 correctly.

This embodiment also includes a handle 238 in the form of a stable rod extending from the rear surface of the casing 205. The handle 238 allows the surgeon to conveniently position the heating device and hold it in position while the orthopedic implant is heated in accordance with the present invention.

In certain embodiments, and with reference to the embodiment shown in FIGS. 20A & 20B, the casing 305 may be modifiable intraoperatively to better conform to the geometry of the implant 102. As shown in FIG. 20A, a transparent biocompatible polymeric casing 305 with a heating element 318 embedded within it is disclosed. Since the casing 305 is transparent, one can see where the heating element 318 resides within the casing 305. Therefore, the surgeon can cut the casing 305 while avoiding damaging the heating element 318. In accordance with an alternative, the casing 305 is an opaque biocompatible polymeric material and has the heating element 318 embedded within it (so the heating element cannot be seen directly). Since the casing is opaque, visible markings 318a are drawn on the outside of the casing 305 to indicate where the embedded heating elements are. This allows the surgeon to avoid cutting through the heating element as they are cutting the casing 305 to their desired size.

This allows the surgeon to trim, bend, or reshape the casing 5, 7 without compromising its protective function, provided the heating element 18 remains undamaged. To facilitate safe modification, the casing 5, 7 may be constructed from transparent materials, such as clear medical-grade polycarbonate or transparent silicone, or may feature visible markings or embedded indicators that delineate the precise location and layout of the heating element 18. These visual guides enable the surgeon to make precise adjustments while avoiding direct interference with the heating element 18.

To ensure versatility, the casing 5, 7 can also be manufactured in a range of standardized sizes and shapes, each optimized to conform to common orthopedic implant geometries used in hip, knee, shoulder, and elbow procedures. These size options accommodate anatomical variation and implant design diversity, enabling the surgeon to select the most appropriate configuration intraoperatively. Custom-fit or pre-contoured casings may also be produced based on imaging data or implant specifications.

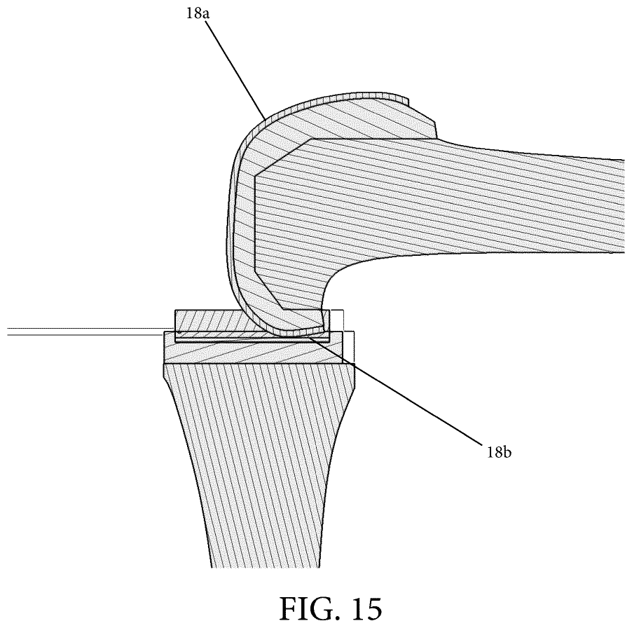

In some configurations, the casing 5, 7 may be shaped to interface with multiple implants simultaneously, such as when heating both femoral and tibial components in a both component knee revision procedure. As illustrated in FIG. 15, a unified casing structure may incorporate multiple heating zones 18a, 18b each tailored to contact distinct implant surfaces while being powered and controlled through a single or modular system.

### Connection

As will be appreciated based upon the following disclosure, connection of the casing to the implant may be achieved in various ways so long as the attachment provides good contact and results in good thermal transfer from the heating element to the implant. For example, the casing can interface with existing attachment mechanisms on the implants, the heating casing can clip or snap to create a mechanical attachment around the implant, the casing can have a geometry which is similar to the implant to create a friction fit to the implant surface, the casing can be attached using adhesive, magnets, clips, etc., the heating casing can be held in place using external pressure, such as hand pressure, Velcro, weighted bags, etc.

With these various connection mechanisms in mind, it is appreciated the casing may be constructed such that the casing contacts a single implant or the casing contacts multiple implants. It is further appreciated that the casing can be deformable to maximize its contact with the exposed implant surfaces or the casing can have a rigid surface that can contact the exposed implant surface.

More specifically, the method of attaching the casing 5, 7 to the implant 102 is a critical aspect of the invention, ensuring that the casing 5,7 maintains consistent and effective contact with the implant surface throughout the procedure. For example, and as mentioned above, several attachment mechanisms can be employed to secure the casing 5, 7 to the tibial component 27 and femoral component 25 of a knee implant, each designed to accommodate different surgical scenarios and implant types.

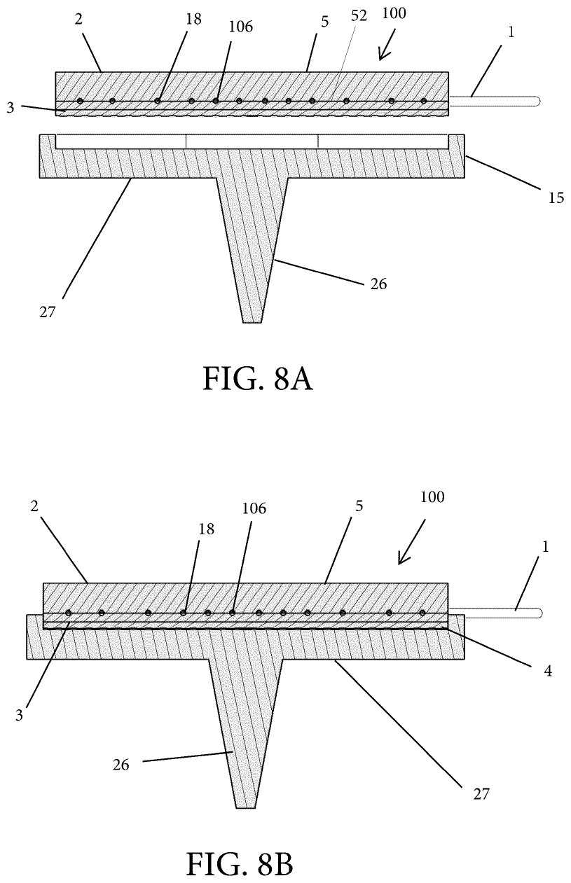

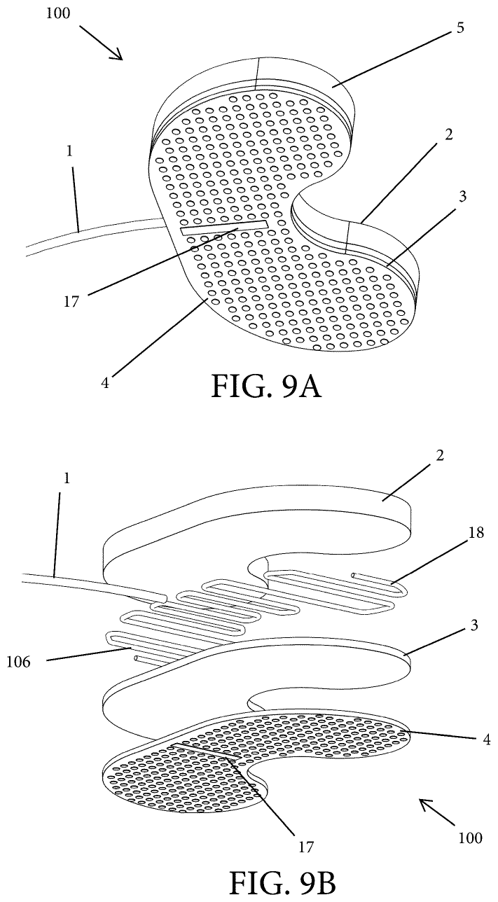

Referring to FIGS. 8A, 8B, 9A, and 9B, one potential attachment method is the use of medical-grade adhesives, such as double-sided tape 4 or biocompatible glue, along the first surface 3 of the casing 5, 7. These adhesives provide a strong bond between the casing 5, 7 and the exposed surfaces 8, 15 of the implant, ensuring that the casing 5, 7 remains securely in place during the heating process. This connection method would be useful for attaching flexible casing 5, 7 to implants with complex surface geometries.

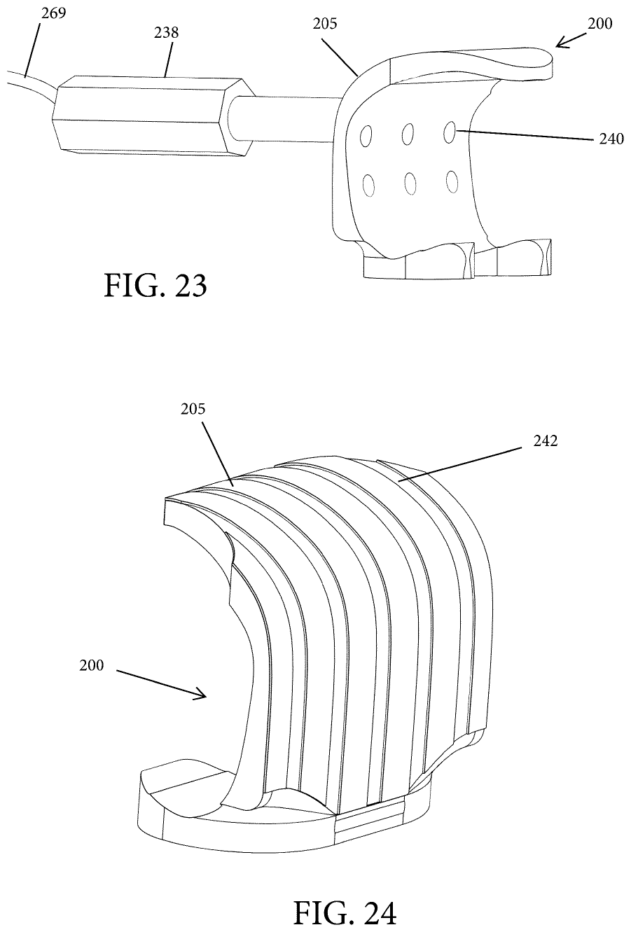

Referring to FIG. 23, magnets 240 offer another effective attachment solution, particularly for metallic implants. Small, powerful magnets 240 embedded within the casing 205 can create a strong magnetic attraction to the implant 102, holding the casing 205 securely in place. This method allows for quick attachment and detachment, facilitating efficient workflow during surgery. The magnets 240 must be carefully positioned to ensure uniform contact between the casing 205 and the implant 102, optimizing heat transfer.

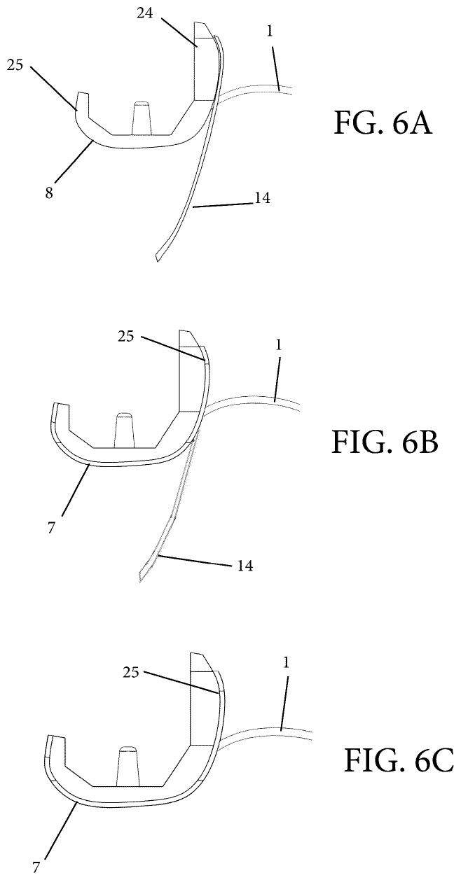

A shape-holding mechanism is another viable option, especially for implants with complex geometries, as shown in FIG. 6A-C. This method involves designing the casing 7 with flexible, shape-memory materials that conform snugly to the implant's surface. The unshaped casing 14 can be shaped to match the implant surface. Once applied, the casing 7 retains its shape, providing consistent contact and heat distribution. Although the embodiment of FIGS. 6A-C is shown with reference to the femoral component, it could certainly be applied to other orthopedic implants, including procedures involving the tibial implant. Additionally, the geometry of the casing such as radius of curvature can be different than the corresponding geometry of the implant so that the shape memory maintains more uniform contact with the implant. These approaches are particularly useful for implants with irregular or curved surfaces, where adhesives or magnets might not provide adequate adhesion. In one embodiment, the casing is constructed from a flexible outer material (e.g., silicone rubber) and includes embedded semi-rigid elements made from a material such as annealed copper, aluminum, or shape-retaining stainless steel wire. These internal supports allow the casing to be bent into shape during placement and hold that configuration once the bending pressure is released, similar to the function of a malleable retractor or twist-tie wire.

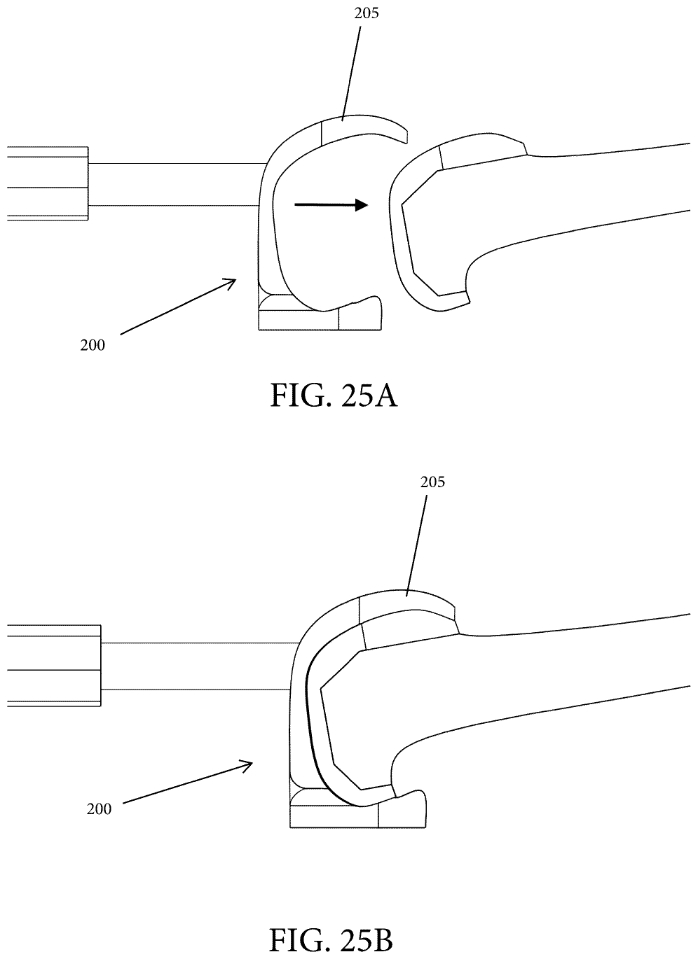

In another embodiment as shown with reference to FIGS. 24, 25A & 25B, the casing 250 is constructed from a semi-rigid material, such as medical-grade polypropylene, polycarbonate, or PEEK, which possesses enough flexibility to deform slightly under manual pressure but sufficient stiffness to “snap” into place once positioned. This configuration is especially effective for implants, for example, with a C-shaped or curved cross-section, such as a femoral component of a knee implant when viewed in a side profile. This embodiment includes the casing 205 with malleable supports 242 (in blue). These supports 242 allow the casing 205 to be formed and shaped when force is applied to it (see FIGS. 25A & 25B showing the application of the heating device to a femoral implant 25). But once the force is removed, the malleable supports 242 will hold the casing in the desired shape.

The casing is pre-formed into a complementary C-shape that mirrors the outer contour of the implant. During application, the two open ends of the casing are gently spread apart, allowing the casing to be placed over the implant surface. Once released, the inherent elasticity and geometry of the casing allow it to snap securely around the implant, similar to how a ball can be snapped into a socket. This snap-fit design ensures stable, uniform contact with the implant and resists unintentional displacement during heating.

For rigid casings 5, precision fitting can ensure secure attachment. The casing 5 can be designed to match the exact dimensions and contours of the implant component, creating a friction and/or mechanical connection that holds the casing firmly in place. Features such as grooves or tabs can be incorporated into the casing design to enhance this fit, preventing any movement during the heating process.

Additionally, mechanical fastening methods, such as clips or clamps, can be employed to secure the casing to the implant 102. These fasteners provide a reliable and adjustable means of attachment, allowing the surgeon to easily position and secure the casing. Mechanical fasteners must be designed to withstand the heat generated by the heating element 18 and ensure that the casing remains stable throughout the procedure.

In some embodiments, the heating device 100, 100′ can be held against the implant surface through externally applied pressure, eliminating the need for adhesives, magnets, or mechanical fasteners. This method is particularly advantageous for temporary or adjustable positioning and for use in situations where traditional attachment mechanisms may be impractical or too time-consuming.

Pressure can be applied manually by the user, typically a surgeon or surgical assistant, who can press the device directly onto the implant surface during the heating cycle. To facilitate safe manual handling at elevated temperatures, the user may wear thermally insulated gloves or use a tool-integrated handle that remains cool to the touch. This method allows for real-time tactile feedback and positional adjustments during the procedure.

Alternatively, external weights can be used to provide consistent downward force. For example, a weighted medical-grade bag can be placed over the casing 5, 7 to press it against the implant surface, maintaining intimate contact and uniform heat transfer. This approach is particularly useful for flat or semi-flat implant surfaces, such as tibial trays or acetabular shells.

Referring to FIG. 23 (as well as the embodiments disclosed with reference to FIGS. 22A & 22B), an embodiment employing a handheld applicator, similar in design to a cattle prod, with a handle 238 at one end and the heating element 218 or flexible casing mounted at the opposite end. The distal end, that is, heating side 203 of the casing 205 may include either a rigid contact plate or a compressible foam pad (as disclosed herein with reference to FIGS. 22A & 22B) that conforms to the curved geometry of the implant 102. The user can press the heating device 200 against the implant 102 using the handle, applying targeted pressure while maintaining a safe distance from the heat-generating surface. The compressible pad enhances surface conformity and heat transfer while reducing slippage.

In additional embodiments, the device may incorporate suction-based attachment, using a soft vacuum ring or internal channel to generate negative pressure that secures the casing to the implant surface. Hybrid configurations are also possible, combining adhesives, magnets, and/or applied pressure for enhanced stability on irregular surfaces. Some casings may include an integrated or detachable handle to facilitate positioning and manual pressure application, after which the handle may be removed, leaving the device in place. In certain designs, temperature-activated adhesives may be used to provide temporary bonding during heating and easy removal afterward. To improve accuracy, the casing 5, 7 may also feature self-retaining contours or mechanical interlocks that engage with standard implant geometries.

### Temperature Sensors

The temperature sensors 17 can be incorporated into the invention, playing a critical role in ensuring precise temperature control and safe operation. These sensors continuously monitor the temperature of the heating element 18 and/or the implant 102, providing real-time data to the control unit 20. This feedback allows the device to maintain the desired temperature range, optimizing the effectiveness of the heating process and preventing overheating. The primary purpose of the temperature sensors 17 is to achieve accurate thermal regulation, which is essential for various surgical applications such as loosening well-fixated implants, clearing bacterial biofilms, and accelerating bone cement curing.

### Type

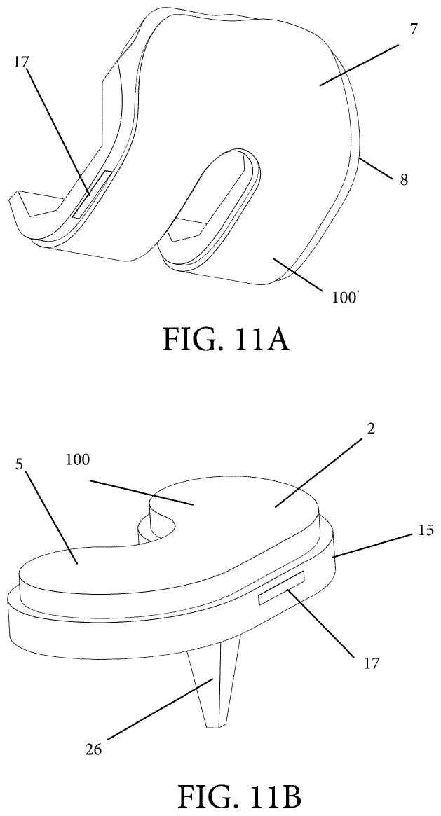

Thermistors are a popular choice for temperature sensing due to their high sensitivity and rapid response time. These components are made from semiconductor materials whose resistance changes predictably with temperature variations. By measuring the resistance of the thermistor, the heating device 100, 100′ can accurately determine the temperature of the heating element 18 and the implant surface. Thermistors can be embedded directly within the heating device 100, 100′ or positioned on the exposed surface 8, 15 of the implant 102, as shown in FIG. 11A-B, to provide real-time temperature feedback. This continuous monitoring allows the control unit to make precise adjustments to maintain the desired temperature range, ensuring effective heat application without risking thermal damage to surrounding tissues.

Thermocouples are another reliable option for temperature measurement. These sensors consist of two different metals joined at one end, creating a junction where a voltage is generated that varies with temperature. The generated voltage can be measured and converted into a temperature reading. They can be placed in direct contact with the implant 102 or integrated into the heating element, providing accurate and stable temperature readings essential for precise thermal management.

Temperature-sensitive stickers offer a simple yet effective method for monitoring temperature. These stickers change color or display a visible indicator when a specific temperature threshold is reached. They can be applied to the exposed surface of the implant or the casing, providing a quick visual confirmation that the desired temperature has been achieved. While less precise than electronic sensors, temperature-sensitive stickers are valuable as an additional safety measure, allowing the surgical team to verify temperature levels at a glance.

Thermal cameras provide advanced, non-contact temperature monitoring by capturing infrared radiation emitted from the implant and its surroundings. These cameras generate thermal images that display temperature variations across the entire surface, enabling comprehensive monitoring of heat distribution. Thermal cameras can detect hotspots and ensure uniform heating, which is crucial for preventing localized overheating and ensuring the effectiveness of the treatment. This technology is particularly useful for visualizing temperature changes in real time, giving surgeons immediate feedback and control over the heating process.

Fiber optic temperature sensors utilize the properties of light to measure temperature changes. These sensors are highly accurate and immune to electromagnetic interference, making them ideal for the surgical environment. They can be embedded within the heating element 18 or placed on the implant surface to provide precise temperature readings. Fiber optic sensors are particularly useful for applications requiring high sensitivity and stability over a wide temperature range.

In some embodiments, multiple temperature sensors 17 may be employed simultaneously to improve accuracy and provide redundant monitoring. These sensors may be positioned at different locations along the heating element 18 (see FIGS. 9A-B), and implant surface (see FIGS. 11A-B) to ensure consistent readings across the treatment area. In the event of sensor failure or discrepancy, the control system may trigger a fail-safe protocol or default to secondary readings to maintain safe operation. All sensors may be subject to pre-use calibration routines or self-diagnostic checks upon startup, ensuring reliable performance throughout the procedure. Temperature data can be displayed in real-time, logged for surgical documentation, or used to adjust power output through a closed-loop PID control system. If temperature readings fall outside the defined safety thresholds, the system can activate visual or audible alerts, prompting immediate corrective action by the surgical team. If the device is verified and validated properly, the heating device 100 would not need to employ temperature sensors at all.

### Control Unit

Referring to FIG. 12, the control unit 20 is the central command center, designed to provide precise and reliable operation of the heating element 18. Its primary purpose is to manage power delivery, regulate temperature, and ensure the safe and effective functioning of the device during orthopedic surgical procedures.

### Interface

The control unit 20 features an intuitive user interface 110, which may combine physical buttons and a touchscreen display. This interface 110 allows surgeons to easily interact with the heating device 100, 100′ setting parameters and monitoring performance. Key controls include a power on/off switch 112, an emergency stop button 114 for immediate shutdown, and a display 116 that provides real-time information on current temperature, set temperature, heating duration, and mode of operation.

### Power Supply

Power management is a critical function of the control unit. The control unit 20, and ultimately the heating device 100, 100′ can be powered by direct wall power 21 (110V AC) or a rechargeable battery. The control unit 20 includes circuitry to manage power distribution, ensuring that the heating element 18 receives a stable and consistent power supply. For battery-operated versions, the control unit 20 monitors battery levels and provides alerts when recharging is necessary, ensuring uninterrupted operation during procedures.

### Modes

The control unit 20 operates in two primary modes: manual mode and preprogrammed heating cycle mode. In manual mode, the surgeon can set specific parameters such as target temperature and duration, allowing for customized operation based on the specific needs of the procedure. This mode provides the flexibility to adjust settings in real-time, accommodating unexpected changes during surgery.

In preprogrammed heating cycle mode, the control unit 20 runs predefined protocols optimized for different types of implants and surgical scenarios. These cycles are designed based on clinical data and best practices, ensuring optimal performance for common applications such as loosening well-fixated implants, clearing bacterial biofilms, and accelerating bone cement 22 curing. Preprogrammed cycles streamline the operation, reducing the need for manual adjustments and minimizing the potential for user error.

The control unit 20 can be configured to recognize different heating element 18 shapes or profiles, allowing compatibility with various implant geometries (e.g., femoral stems, acetabular cups, tibial trays). Upon connecting a specific element, the system can auto-load the corresponding protocol or allow the surgeon to select from a library of preconfigured heating profiles.

### Temperature Regulation

Temperature regulation is a crucial aspect of the control unit's functionality. The control unit 20 may control the power to the heating element 18 using a precise feedback loop. This system continuously adjusts the power output to maintain the target temperature, responding to real-time feedback from temperature sensors 17. These temperature sensors 17, such as thermistors or thermocouples, are strategically placed within or around the heating element 18 to provide accurate temperature readings.

Safety features are integrated into the control unit 20 to protect both the patient and the device. Overheat protection mechanisms automatically reduce or cut off power if the temperature exceeds a predefined threshold, preventing thermal injury to surrounding tissues. The control unit 20 also provides audible and visual alerts for critical events, such as reaching target temperature, low battery, or sensor malfunctions, enabling the surgical team to respond promptly to any issues.

In addition, the control unit 20 works in conjunction with the heating elements 18, provide for uniform heating element distribution or varying density of the heating element distribution depending on implant geometry or variable heating profile.

### Data

The control unit 20 may process the data from these sensors, comparing the current temperature to the set target. If the temperature deviates from the desired range, the control unit 20 adjusts the power output to the heating element 18, either increasing or decreasing it to achieve the target temperature. This precise control ensures that the heating element 18 operates within safe and effective temperature ranges, preventing overheating and ensuring consistent thermal treatment.

Additionally, the control unit 20 stores and logs operational data, which can be useful for post-surgical analysis and device maintenance. This data includes temperature profiles, duration of heating cycles, and any alerts or errors encountered during operation. Access to this information allows for the continuous improvement of surgical techniques and device performance.

### Connections

The control unit 20 connects to the heating element 18 via a robust and secure electrical interface, ensuring reliable power transmission and data communication between the two components. This connection is typically established through a high-quality, medical-grade connector 19 that is both durable and easy to use. To facilitate versatility and ease of use, the connection is designed to be detachable, allowing the heating element 18 to be easily connected or disconnected from the control unit as needed. This detachment feature is particularly useful for sterilization and maintenance purposes, as it enables each component to be separately cleaned and inspected without compromising the integrity of the connection. Additionally, the detachable design allows for quick replacement or interchangeability of heating elements 18, enhancing the device's adaptability to different surgical requirements and improving overall workflow efficiency in the operating room.

The wire 1 used to connect the control unit to the heating element 18 is a high-quality, medical-grade cable designed for durability, flexibility, and safety. It is insulated with biocompatible, heat-resistant materials to withstand the high temperatures generated during operation and to prevent any electrical interference or hazards. The cable 1 is also designed to be flexible, allowing for easy maneuverability and positioning during surgical procedures without compromising the integrity of the connection. The portion of cable 1 on the surgical field can remain sterile while the portion out of the surgical field is not.

### Multiple Heating Elements

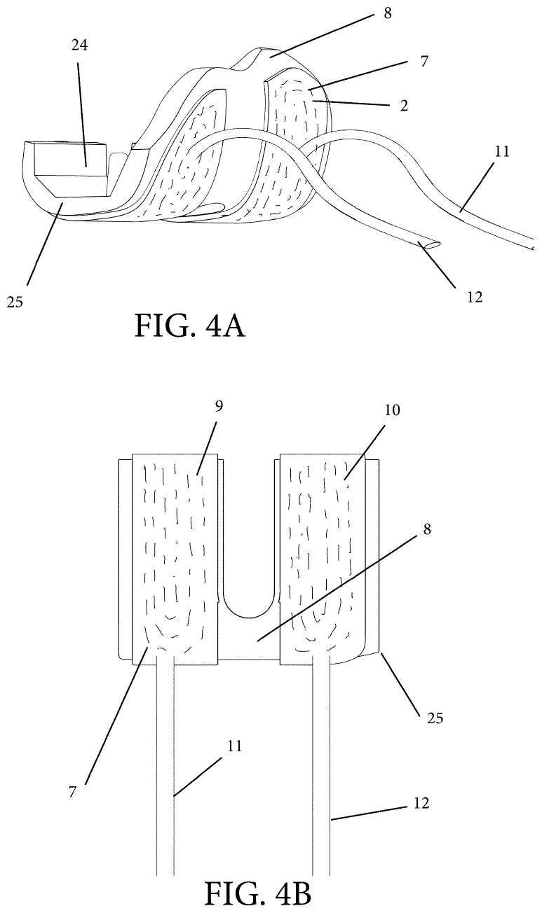

The control unit 20 is designed to accommodate the simultaneous use of multiple heating elements 9, 10 (for example, as shown with reference to the embodiment disclosed in FIGS. 4A and 4B), enhancing the versatility and efficiency of this invention. This capability is particularly advantageous in complex surgical procedures where multiple implants or larger surface areas require heating. In accordance with such usage, the control unit 20 features multiple heating element wires 1, 11, 12, each capable of independently controlling a separate heating element 9, 10, 18. These ports are equipped with high-quality, medical-grade connectors 19 that ensure secure and reliable connections for each heating element.

When multiple heating elements 9, 10 are in use, the control unit 20 employs advanced algorithms to manage and balance the power distribution, ensuring each element receives the appropriate amount of power to maintain its target temperature. The control unit 20 monitors and regulates the temperature of each heating element 18 individually, using real-time feedback from temperature sensors 17 embedded in or around each element. This independent control allows for precise temperature management across different areas, optimizing the heating process and ensuring uniform thermal treatment.

As discussed above, the heating device may be constructed to apply heat to multiple surfaces. In particular, and with reference to FIGS. 16 and 17A-17F, an embodiment of a heating device 200 with a casing 205 incorporating multiple heating zones, each tailored to contact distinct implant surfaces while being powered and controlled through a single or modular system, is disclosed. The disclosed embodiment is specifically designed for heating both femoral and tibial components in a knee revision procedure. However, it is appreciated that the concepts underlying this disclosed embodiment may be applied to other procedures.

As with the embodiment disclosed above, the heating device includes a heating element (in particular, a first heating element 218a and a second heating element 218b) designed to deliver controlled and uniform heat to the femoral and tibial components and the implant. The heating elements are constructed in any of the manner discussed above.

The first heating element 218a and the second heating element 218b are housed within a casing 205 shaped and dimensioned to contact both the tibial component 27 and the femoral component 25. The casing body 219, therefore, includes a first member 221 shaped and dimensioned for contact with the tibial component 27 during the heating procedure and a second member 223 shaped and dimensioned for contact with the femoral component 25 during the heating procedure. The first member 221 and the second member 223 are integrally formed to define the casing body 219.

The first member 221 is substantially rectangular in shape and includes an exposed lower wall 225, an upper wall 227 that is integral with the second member 223, a first side wall 229, a second side wall 231, a third side wall 233, and a fourth side wall 235. The first, second, third, and fourth side walls 229, 231, 233, 235 extend between the exposed lower wall 225 and the upper wall 227. Within the first member 221 and positioned between the exposed lower wall 225 and the upper wall 227 is a cavity 237 in which the first heating element 218a, as well as other functional components, are housed.

The second member 223 is substantially C-shaped and includes a lower portion 239 that is integrally formed with the upper wall 227 of the first member 221. The second member 223 also includes a concave internal wall 241 and a convex external wall 243. Extending between the concave internal wall 241 and the convex external wall 243 are a first lateral side 245, a second lateral side wall 247, a first end side wall 249, and a second end side wall 251. Within the second member 223 and positioned between the concave internal wall 241 and the convex external wall 243, is a cavity 253 in which the second heating element 218b, as well as other functional components, are housed.

In practice, the heating device 200 of this embodiment is utilized for applying resistive heat to an orthopedic implant in the following manner. Briefly, and as is discussed below in greater detail, exposed implant surfaces are first accessed. A retractor instrument 120 may be used to aid in exposure of the orthopedic implant and protection of surrounding soft tissues. The heating device 200 is then attached to the exposed implant surface and the heating device 200 is activated. The method also includes using the control unit 20 to provide precise and reliable operation of the heating element of the heating device by managing power delivery, regulating temperature, and ensuring the safe and effective functioning of the device during orthopedic surgical procedures. Where desired temperature sensors 17 are positioned on the orthopedic implant and/or modular components of the orthopedic implant may be removed to fully expose underlying implant surfaces.

More specifically, first, the tibial tray of the tibial component 27 is removed. Thereafter, the heating device 200 is forced between the tibial component 27 and the femoral component 25. In particular, the exposed lower wall 225 is positioned to contact the exposed upper surface of the tibial component 27 and the concave internal wall 241 of the second member 223 is forced over the exposed surface of the femoral component 25. The concave internal wall 241 of the second member 223 is shaped to slide over the exposed surface of the femoral component 25 and snap into position. As such, the concave shape of the concave internal wall 241 is such that the opening defined thereby is slightly smaller than the diameter of the femoral component 25, but the second member 223 exhibits resilience allowing it to flex and fit over the femoral component 25, ultimately snapping into place to securely possession the concave internal wall 241 in direct contact with the exposed surface of the femoral component 25. It is, however, appreciated that the diameter of the concave internal wall 241 could be the same as the diameter of the femoral component, given that the concave internal wall 241 has a lip/edge to grab on to.

In practice, it is appreciated that an insertion paddle 255 may be utilized to assist in the deployment of the heating device 200 between the tibial component 27 and the femoral component 25. In accordance with a disclosed embodiment, the insertion paddle 255 is an elongated member having a first end 257 shaped and dimensioned for engagement by the medical practitioner performing the procedure, and a second end 259 shaped and dimensioned for engagement with the third side wall 233 of the first member 221.

Once the heating device 200 is positioned in the desired manner, a secure pressure pad 261 is secured about the knee of the patient. In accordance with the disclosed embodiment, the pressure pad 261 includes a rectangular pad member 263, as well as first and second Velcro straps 265, 267 shaped and dimensioned for encircling the leg of the patient above and below the heating device 200. In accordance with another embodiment, the straps are not used and the surgeon manually applies pressure to the heating device 200 via the handle 38 on the back of the cuff (see FIGS. E1 and E2).

With the heating device 200 in position, the control unit 220 is connected to the heating device 200 via a cable 269 that is plugged that connects the heating device 200 to the control unit 220. The cable 269 includes a first end 271 selectively coupled to the control unit 220 and a second end 273 selectively coupled to the input 271 of the heating device 200. The second end 273 of the cable 269 includes a grip or handheld flange 275 to facilitate convenient attachment of the second end 273 to the input 271 of the heating device 200.

Once the procedure is completed, the heating device 200 is removed by reversing the steps previously discussed.

### Methods

The invention and method are designed to be versatile and applicable to various types of implants, including knee, hip, shoulder, elbow implants, and fixed implants, such as rods, screws, and plates. The following is an example procedure using a knee replacement implant, though the principles and steps can be adapted to other implant types.

### Gaining Access to the Exposed Implant Surfaces

Once the limb is properly positioned, the surgeon proceeds to make the incision. The type and location of the incision depend on the specific surgical approach and the location of the implant. A surgical approach is performed.

With the knee joint exposed, the surgeon proceeds to clean away any debris, fluid, or soft tissue that may obscure the implant. Retractor instruments may be placed to aid in the appropriate exposure of the implants and protection of surrounding soft tissues. This step is crucial for ensuring that the heating device can make direct contact with the implant surface. Any fibrous tissue, bone fragments, or other debris are meticulously removed using surgical instruments such as rongeurs, curettes, and suction devices.

If the implant includes modular components, such as a polymer tibial liner, these are carefully removed to fully expose the underlying implant surfaces. Once the implant surfaces are fully exposed, the surgeon may perform further irrigation and debridement of the surgical site. The surgeon may then dry the implant. This may involve using sterile gauze, suction devices, or specialized drying instruments to remove any residual fluids.

### Attaching the Heating Element to the Exposed Surfaces of the Implant

With the surgical site prepared and the implant surfaces fully exposed, the next step is to attach the casing containing the heating element to the exposed surfaces of the knee implant. This process may involve using both rigid and flexible casings, depending on the specific components of the knee implant being treated.

For the tibial component of the knee implant, a rigid casing can be used. The casing is designed to match the overall shape and size of the polyethylene tibial tray, allowing it to maximize the surface contact with the tibial implant. This precise fit ensures that the casing remains stable and in full contact with the implant surface throughout the heating process.

To attach the rigid casing, the surgeon aligns it with the tibial component, ensuring that it interfaces correctly, just as the original polyethylene liner would. The rigid casing may include grooves or tabs that help secure it in place, preventing any movement during the procedure. This design allows for easy attachment and detachment, facilitating a smooth workflow.

For the femoral component of the knee implant, a flexible casing is utilized to accommodate the curvature and complex geometry of the implant surface. The flexible casing can be bent and contoured to match the shape of the femoral implant, ensuring comprehensive coverage and effective heat transfer.

The process begins with the surgeon carefully bending the flexible casing to fit the curvature of the femoral component. The flexible material allows for precise adjustments, ensuring that the casing conforms to the implant surface without gaps. In some cases, the flexible casing may come pre-shaped if it is made from a material that can hold its shape after being bent. This pre-shaping simplifies the attachment process, reducing the time required to achieve a perfect fit. The surgeon may also modify the shape of the casing intraoperatively to achieve a better fit.

Before final attachment, the surgeon performs a test run by positioning the casing on the implant without securing it. This allows for any necessary adjustments to ensure optimal contact and alignment. Once the surgeon is satisfied with the fit, the casing is securely attached using any suitable attachment method including, but not limited to, those described below.

If medical-grade adhesives are used, the surgeon first ensures that the implant surface is clean and dry. The surgeon exposes the layer of adhesive tape on the underside of the casing. The casing is carefully positioned onto the implant, and firm pressure is applied to ensure a secure bond. The surgeon checks for any gaps or misalignments, making adjustments as needed. The adhesive provides a strong bond that holds the casing in place throughout the heating process.

If using magnets, the surgeon ensures that the implant is free of debris that might interfere with magnetic adhesion. The casing, embedded with small, powerful magnets, is then positioned over the implant. The magnets create an immediate and strong attraction to the metallic implant, securing the casing in place. The surgeon verifies that the casing is correctly aligned and making full contact with the implant surface, ensuring consistent heat transfer during the procedure.

For shape-holding mechanisms, the surgeon begins by manually contouring the flexible casing to the shape of the implant. This involves bending and shaping the casing until it fits snugly against the implant surface. Once the desired shape is achieved, the casing is applied to the implant, where it retains its shape due to the material's memory properties. The casing may come pre-contoured for the specific implant application. The surgeon ensures that the casing remains in place and provides uniform contact with the implant, optimizing heat transfer.

If mechanical fasteners are used, the surgeon positions the casing over the implant and uses clips, clamps, or similar fasteners to secure it. The fasteners are carefully applied to avoid damaging the implant or casing while ensuring a tight fit. The surgeon may adjust the fasteners to achieve the best possible contact between the casing and the implant surface. The mechanical fasteners hold the casing firmly in place, preventing any movement during the heating process.

In cases where the implant has extensive exposed surfaces, multiple casings can be used simultaneously to ensure comprehensive coverage. Each casing can be independently contoured and attached to different areas of the implant, providing a customized and effective heating solution. The control unit can manage multiple heating elements, ensuring that each casing operates within the desired temperature range. Thermal paste can be used to fill air gaps or voids between the casing and the implant.

### Positioning Temperature Sensors

Temperature sensors, such as thermistors or thermocouples, may play a critical role in ensuring precise temperature control. These sensors can be strategically placed within or around the heating element and the implant to provide real-time temperature feedback to the control unit. They can also be sandwiched between the casing and the exposed surfaces of the implant.

The installation of the temperature sensors begins with securing them in positions that accurately reflect the temperature of the implant surfaces being heated. For example, sensors may be embedded within the casing of the heating element or attached directly to the implant using medical-grade adhesive. These sensors are small and minimally invasive, ensuring they do not interfere with the surgical procedure or the heating process.

Once the sensors are positioned, they are connected to the control unit. The connections are secure yet detachable, allowing for easy removal and replacement as needed.

### Activating the Control Unit

Once the casing is securely attached to the exposed surfaces of the knee implant, the surgeon proceeds to select the appropriate heating program on the control unit. This step is crucial for ensuring that the heating element operates within the optimal temperature range and duration required for the specific surgical procedure. The control unit offers both manual mode and pre-programmed cycles, providing flexibility and precision in temperature management.

In manual mode, the surgeon has the flexibility to customize the heating parameters according to the specific needs of the procedure. The control unit interface features a combination of physical buttons and a touchscreen display, making it user-friendly and intuitive.

To begin, the surgeon powers on the control unit and selects “Manual Mode.” Once in manual mode, the surgeon can set the target temperature. The control unit displays the current temperature, and the surgeon can monitor it in real-time.

The surgeon next sets the duration for which the heating element should maintain the target temperature. The surgeon can choose to activate real-time temperature feedback by selecting the appropriate option on the control unit. This feature allows the device to continuously adjust the power output to the heating element, ensuring precise temperature control based on feedback from embedded temperature sensors.

After configuring the settings, the surgeon initiates the heating process by pressing the “Start” button. The control unit continuously displays the current temperature, target temperature, and elapsed time, allowing the surgeon to monitor the process closely. If any adjustments are needed during the procedure, the surgeon can easily modify the settings via the touchscreen interface.

For standard procedures, the control unit offers pre-programmed heating cycles that have been optimized based on clinical data and best practices. These cycles simplify the process by providing preset parameters tailored for specific surgical applications, such as loosening well-fixated implants, clearing bacterial biofilms, and accelerating bone cement curing.

The surgeon selects the appropriate cycle for the procedure. For example, if the goal is to loosen a well-fixated knee implant, the surgeon might choose the “Implant Loosening” cycle. The control unit automatically loads the preset parameters, including the target temperature and duration, optimized for reaching the glass transition temperature of the bone cement (typically between 60° C. and 140° C.).

The pre-programmed cycle also incorporates real-time temperature feedback, allowing the control unit to adjust the power output to maintain the target temperature precisely. This feature ensures consistent and effective heating throughout the procedure.

Once the cycle is selected, the surgeon presses the “Start” button to initiate the heating process. The control unit displays the current temperature, target temperature, and remaining time, providing continuous monitoring throughout the cycle. If necessary, the surgeon can pause or stop the cycle using the control unit's controls.

The duration for which the implant is heated is critical to not damaging surrounding tissues. The heating process begins by targeting the surface of the implant, allowing the generated heat to initially warm the exposed surfaces. This heating phase is typically maintained for a period between about 5 seconds and about 15 minutes, depending on the size and material of the implant.

Once the surface of the implant reaches the desired temperature, time may be needed for the heat to diffuse throughout the remaining parts of the implant. This diffusion process ensures that the non-exposed surfaces of the implant is also sufficiently heated.

### Loosening Well-Fixed Implants

The device operates by heating the implant to a temperature that effectively disrupts the implant-bone, bone-cement, and/or implant-cement interfaces, facilitating easier removal. This process is particularly beneficial for well-fixated implants, where the bond between the implant and the surrounding bone or cement is strong and requires careful management to avoid excessive force and potential damage.

The glass transition temperature of polymethyl methacrylate (PMMA) bone cement typically ranges between 60° C. and 140° C. This temperature range represents the point at which the bone cement transitions from a hard and brittle “glassy” state to a more pliable and viscous or rubbery state, significantly weakening its mechanical properties. To achieve this transition, the heating element generates temperatures that may be higher than the implant surface itself due to the nature of energy transfer between the heating element and the implant. The predetermined temperature for the exposed surface of the implant is generally between 100° C. and 160° C., depending on the heating duration.

The heat transfer properties of PMMA bone cement play a critical role in ensuring patient safety. PMMA has relatively low thermal conductivity, which means that the heat generated in the implant is not easily transferred to the surrounding bone and tissues. This characteristic helps to confine the heating effect to the implant and its immediate interface, minimizing the risk of thermal damage to the surrounding biological structures. By maintaining the temperature within the appropriate range and taking advantage of the thermal properties of PMMA, the device ensures effective disruption of the implant-bone and implant-cement interfaces without compromising the integrity of the surrounding tissues.

Once the implant reaches the desired temperature, the device should be promptly removed from the implant. At this point, the surgeon should proceed with the standard procedure to remove the implant. The heating process disrupts the bone-implant or implant-cement interfaces, making it easier to extract the implant with minimal force. If the surgeon encounters more resistance than expected, they have the option to reheat the implant to further weaken these interfaces, ensuring a smoother removal process.

In some cases, it may be beneficial to attach implant removal tools, such as a slap hammer, mallet, or extraction pliers, to the implant while it is being heated. This allows the surgeon to apply gentle force as the implant is reaching or maintaining the elevated temperature. In other cases, the surgeon can use a bone tamp and mallet or similar tools to remove the implant once heated. The combination of heat and mechanical assistance can be highly effective in loosening well-fixated implants.

Throughout the process, the user can monitor the temperature of the implant and surrounding tissue using thermal cameras or temperature sensors. This ensures that the implant is heated effectively without causing damage to adjacent structures.

In situations where the implant remains resistant to removal after initial heating, repeated heating cycles combined with incremental mechanical force can gradually weaken the interfaces, facilitating easier extraction.

### Reducing Bacterial Load

This invention is also designed to effectively clear bacterial biofilm and reduce bacterial burden from the surface of implants. Bacterial biofilms, which are complex communities of bacteria encased in a protective extracellular matrix, pose a significant challenge in orthopedic surgeries due to their resistance to standard antibiotic treatments and cleaning methods. The application of controlled heat disrupts these biofilms, enhancing the effectiveness of subsequent antimicrobial treatments and improving patient outcomes.

The intraoperative application of this heating device offers a powerful adjunctive strategy for managing orthopedic implant infections, with significant benefits for patient outcomes. One of the most impactful uses of the device is its potential to reduce the need for implant explantation. In many cases, particularly with early or localized infections, removing a well-fixed implant solely due to infection can lead to unnecessary surgical trauma, increased recovery time, long-term functional impairment, and need for further surgical procedures.

By applying localized, controlled heat to the exposed surfaces of the implant, the device disrupts bacterial biofilms that would otherwise shield the infection from immune response and systemic antibiotics. This disruption exposes the underlying bacteria, making them more susceptible to antimicrobial agents. Heat weakens the protective extracellular polymeric substance (EPS) matrix and increases bacterial membrane permeability, thereby enhancing antibiotic penetration and effectiveness. As a result, systemic or locally applied antibiotics can act more efficiently, even in areas previously protected by the biofilm barrier. Heat application is also bactericidal, leading to decreased bacterial burden at the time of the procedure.

Implant-associated infections can be classified as acute or chronic, each requiring different thermal treatment strategies. Acute infections typically occur within days to weeks of surgery and are often limited to exposed implant surfaces. These early-stage infections involve less mature biofilms with a weaker extracellular matrix, making them more susceptible to heat disruption. In such cases, direct application of the heating device to the accessible implant surface is typically sufficient, requiring lower temperatures and shorter heating durations.

Chronic infections develop over longer periods and involve mature, antibiotic-resistant biofilms that may extend into non-exposed implant surfaces, such as areas embedded in bone or cement. These deeper infections require longer heating durations and potentially higher surface temperatures to allow sufficient thermal diffusion into the non-exposed regions. The heating device's flexible temperature control and surface conformity enable it to target both superficial and deeper infections, enhancing biofilm disruption and improving treatment outcomes in both acute and chronic cases.

The heating element is set to a temperature that is bactericidal and effective in disrupting the biofilm matrix, typically between 40° C. and 120° C. This temperature range is sufficient to break down the extracellular polymeric substances (EPS) that protect the bacteria within the biofilm, making them more susceptible to antibiotic treatment and mechanical removal. The heating element may need to operate at slightly higher temperatures than the implant surface to ensure the effective transfer of heat through the implant material to the biofilm. The heat is not only useful in disrupting biofilm but can also reduce bacterial burden on the implant. Heat application is a widely utilized method to eradicate bacteria. Lower temperature exposure between 40-60° C.) may weaken the EPS structure without fully eradicating the underlying bacteria, making this step ideal for use alongside targeted antibiotics. Higher temperatures above 60° C., if used briefly and within safety margins, may exert direct bactericidal effects while still preserving adjacent tissue integrity.

The duration of heating is critical to ensuring the disruption of the biofilm without causing thermal damage to surrounding tissues. The heating process usually begins with an initial phase of about 5 seconds to 15 minutes, depending on the extent of the biofilm and the size/geometry of the implant. The heating element can be cycled on and off to allow the heat to penetrate the biofilm effectively while preventing overheating of the implant surface.

After the initial heating phase, the biofilm structure is disrupted, making the bacteria more vulnerable to antibiotic/antiseptic solutions and mechanical treatments. The surgeon can then mechanically debride the implant surfaces further and/or apply a targeted antibiotic/antiseptic solution therapy directly to the implant surface. The heat treatment not only disrupts the biofilm but also enhances the penetration of antibiotics and antiseptic treatments, ensuring more effective bacterial eradication. If residual biofilm remains after initial treatment, the device may be reapplied in subsequent cycles, allowing for incremental bacterial reduction without tissue trauma.

In some cases, additional mechanical debridement may be performed to remove the disrupted biofilm and reduce residual bacterial burden. The combination of heat, antibiotics/antiseptic treatments, and mechanical cleaning provides a comprehensive approach to biofilm management, significantly reducing the risk of persistent or recurrent infections. Any remaining debris or disrupted biofilm can be removed using standard surgical tools, ensuring a clean and infection-free implant surface.

In addition to intraoperative applications, the device can be utilized to preheat orthopedic implants prior to insertion as a means of reducing the risk of implant-associated infections. Even with standard sterilization protocols, orthopedic implants may retain trace levels of contaminants or be exposed to environmental bacteria during handling and setup. Preheating the implant surface immediately before insertion provides an additional antimicrobial safeguard.

The heating device can be applied directly to the implant on the surgical tray, using either a flexible or rigid heating element that conforms to the implant geometry. The implant is heated to a predetermined temperature, typically between 60° C. and 120° C., for a controlled duration sufficient to inactivate residual bacteria or disrupt any early biofilm formation that may have occurred during handling. The selected temperature and duration are optimized to maximize antimicrobial effect without altering the implant's structural or mechanical properties.

This preheating process can be conducted outside the surgical field, minimizing risk to the patient and allowing the implant to be promptly transferred into the operative site after the heating cycle is complete. In some cases, the implant may be cooled slightly before insertion to avoid thermal irritation of surrounding tissues.

### Curing Bone Cement

The invention and method are also designed to accelerate the curing process of bone cement, ensuring a stable and secure fixation of the implant. This method is particularly valuable in reducing surgical time, improving the initial mechanical stability of the implant, and ensuring proper implant alignment while cement curing occurs.

Once the bone cement is applied and the implant is positioned, the surgeon takes special care to ensure that the implant remains immobile throughout the heating cycle. Maintaining the position of the implant during cement curing is crucial to prevent any displacement or misalignment that could compromise the cement bond and overall implant stability.

In some cases, it may be necessary to heat individual components of the knee implant separately. Real-time temperature feedback from sensors ensures that the heating element operates within the precise temperature range required for curing the bone cement, typically between 40° C. and 100° C. This targeted heating accelerates the exothermic polymerization reaction of the bone cement, reducing the time needed for it to harden and bond effectively to the implant and bone.

For a more integrated approach, both the tibial and femoral components of the knee implant can be heated simultaneously. After the bone cement is applied and both components are in place, the knee is extended so that the tibial and femoral components are applying force on each other, simulating the natural load-bearing condition of the joint. This ensures that the bone cement cures under optimal pressure conditions, promoting a stronger bond and conformation to the bony surfaces. It also ensures adequate implant alignment during the cement curing process.

In this scenario, the heating element, designed in the shape of the polyethylene for the tibial tray, is inserted into the tibial component. As the knee is extended and the components press against each other, the heat from the tibial heating element begins to spread to both the tibial and femoral components. This simultaneous heating ensures that both components are uniformly heated, allowing the bone cement to cure more efficiently and evenly across the entire joint interface. The heating element in the shape of the tibial tray could also have a flexible or rigid portion that simultaneously contours along the anterior femoral component and heats this region. This would promote uniform implant heating and more universal cement curing.

After the heating cycle is complete, the surgeon verifies that the bone cement has fully cured and that the implant is securely fixed. This may involve visual inspection, palpation, or using diagnostic tools to assess the stability and integrity of the cement bond.

In addition to intraoperative applications, the device can be utilized to preheat orthopedic implants prior to insertion as a means to expedite the cement curing process. In this scenario, the tibial and femoral components may be heated prior to implantation. The elevated implant temperature in the proximity of the bone cement expedites the cement curing process.

### Clean-Up & Disposal

After the heating process is complete, the next critical step is to safely and effectively remove the casing and heating elements from the implant. This process ensures that the implant, if it is to remain within the body, is clean and free of any residues, and that the heating elements are properly handled for either disposal or sterilization.

To begin the removal process, the surgeon first ensures that the heating element has sufficiently cooled to a safe temperature to handle. Using sterile gloves and instruments, the surgeon gently detaches the casing from the implant. If medical-grade adhesives were used, they may require careful peeling or the application of a solvent to dissolve the adhesive without damaging the implant surface. Magnets can be easily detached by gently pulling the casing away from the implant, ensuring that the magnetic attraction is overcome without abrupt movements that could displace the implant. Shape-holding mechanisms and mechanical fasteners are also disengaged with care to avoid any undue pressure on the implant.

If the implant is to remain within the body, as in the cases of cement curing or infection treatment, thorough cleaning and disinfection of the implant are essential. The surgeon uses sterile saline solution or appropriate disinfectants to rinse the implant surface, removing any residues left by the casing or heating process. Specialized brushes or swabs can be used to ensure all traces of adhesive or debris are removed, paying close attention to crevices and hard-to-reach areas. In the scenario of infection treatment, further antibiotic/antiseptic solutions can be applied to the surgical site as are currently typically utilized in this surgery.

Once the casing is removed, the heating element itself is detached from the control unit. This is typically done by disconnecting the insulated, medical-grade cables that link the heating element to the control unit. The connectors are designed to be easily detachable, allowing for quick and efficient disconnection.

If the heating element and casing are designed for single use, they should be disposed of according to medical waste disposal protocols. For reusable heating elements and casings, the components must undergo thorough cleaning and sterilization before being used in subsequent procedures.

While the preferred embodiments have been shown and described, it will be understood that there is no intent to limit the invention by such disclosure, but rather, is intended to cover all modifications and alternate constructions falling within the spirit and scope of the invention.

1. A system for heating orthopedic implants, comprising:
a heating device, comprising:
a heating element;
a casing supporting or housing the heating element, the casing being shaped and dimensioned for engagement with an orthopedic implant; and
a control unit providing precise and reliable operation of the heating element by managing power delivery, regulating temperature, and ensuring safe and effective functioning orthopedic surgical procedures.

2. The system for heating orthopedic implants according to claim 1, wherein the heating element comprises a resistance heating wire.

3. The system for heating orthopedic implants according to claim 1, further including a temperature sensor(s).

4. The system for heating orthopedic implants according to claim 1, wherein the heating element operates at (1) a temperature of 100° C. to 150° C. to heat the orthopedic implant to disrupt the mechanical bond of bone cement or non-cemented implant fixation; (2) at a temperature of 40° C. to 120° C. decrease bacterial burden and disrupt the biofilm; and/or at a temperature of 40° C. to 100° C. for curing the bone cement.

5. The system for heating orthopedic implants according to claim 1, wherein the casing is adapted to be trimmed or modified to a desired shape which will not disrupt or damage the heating element.

6. The system for heating orthopedic implants according to claim 1, further including a handle extending from the casing allowing a user to conveniently position the heating device upon the orthopedic implant.

7. The system for heating orthopedic implants according to claim 1, wherein the casing incorporates multiple heating element geometries, each tailored to heat distinct implant surfaces.

8. The system for heating orthopedic implants according to claim 7, wherein the casing is structured for simultaneously heating both femoral and tibial components in a knee revision procedure.

9. The system for heating orthopedic implants according to claim 7, wherein the heating element includes a first heating element and a second heating element, the first heating element and the second heating element are housed within a casing shaped and dimensioned to contact both a tibial component and a femoral component.

10. The system for heating orthopedic implants according to claim 9, wherein the casing includes a casing body that comprises a first member shaped and dimensioned for contact with the tibial component and a second member shaped and dimensioned for contact with a femoral component.

11. The system for heating orthopedic implants according to claim 10, wherein the first member is substantially rectangular in shape and the second member is substantially C-shaped.

12. A heating device for heating orthopedic implants, comprising:
a heating element comprising a resistance heating wire, the heating element includes a first heating element and a second heating element; and
a casing supporting or housing the heating element, the casing including a casing body comprising a first member and a second member shaped and dimensioned for engagement with an orthopedic implant, the first heating element and the second heating element are respectively housed within the first member and the second member.

13. The heating device according to claim 12, wherein the first member is shaped and dimensioned for contact with a tibial component and the second member is shaped and dimensioned for contact with a femoral component.

14. The heating device according to claim 12, further including a temperature sensor.

15. The heating device according to claim 12, wherein the first member and the second member of the casing define multiple heating zones, each tailored to contact distinct implant surfaces.

16. The heating device according to claim 12, further including a handle extending from the casing allowing a user to conveniently position the heating device upon the orthopedic implant.

17. The heating device according to claim 12, wherein casing is structured for simultaneously heating both femoral and tibial components in a knee revision procedure.

18. The heating device according to claim 17, wherein the casing the first member is shaped and dimensioned for contact with the tibial component and the second member is shaped and dimensioned for contact with a femoral component.

19. A system for heating orthopedic implants, comprising:
an orthopedic implant;
a heating device, comprising:
a heating element; and
a casing supporting or housing the heating element, the casing being shaped and dimensioned for engagement with an orthopedic implant; and
a control unit providing precise and reliable operation of the heating element by managing power delivery, regulating temperature, and ensuring safe and effective functioning of the heating device during orthopedic surgical procedures.

20. A method of applying resistive heat to an orthopedic implant comprising:
accessing an exposed implant surface;
applying a retractor instrument to aid in exposure of the orthopedic implant and protection of surrounding soft tissues;
attaching a heating device to the exposed implant surface; and
activating the heating device.

21. The method according to claim 20, further including removing modular components of the orthopedic implant to fully expose underlying implant surfaces.

22. The method according to claim 20, further including a control unit providing precise and reliable operation of a heating element of the heating device by managing power delivery, regulating temperature, and ensuring safe and effective functioning during orthopedic surgical procedures.

23. The method according to claim 22, wherein the heating device further includes temperature sensor(s), and the temperature sensor(s) are positioned on the orthopedic implant and/or the casing.

24. The method according to claim 23, wherein the control unit heats the heating element to apply sufficient heat to the non exposed implant surface so that the bone cement or bone-implant interface reaches a temperature between 100° C. and 150° C.

25. The method according to claim 24, wherein the control unit heats the heating element to apply sufficient heat to heat an exposed surface of the orthopedic implant to between 100° C. and 160° C. for reaching a glass transition temperature of polymethyl methacrylate (PMMA) bone cement.

26. The method according to claim 25, wherein the control unit heats the heating element to apply sufficient heat to heat the orthopedic implant to 40° C. and 100° C. to reduce bacterial load from the surface of orthopedic implants.
